SEQUENCE LISTING (10 sequences)

SEQ ID NO: 1 - DNA, 1371 bp, Nicotiana tabacum
  gene (61)..(978): NtCPIP1
gcaaaagagcgagagagagagagagagagagagagagagagagagagagagagagagaaaatgggcgttgattactacaaaatactcaaagtgtcacgcaatgcgagtgaagaagatttgaaaaaatcgtacaagcgattggcgatgaaatggcatccggataagaacagtgagaaagaagcagaagcgaaattcaagcagatatcggaggcctatgatgtgctaagtgatccacagaagcgtcagatctatgatatatacggcgatgaggcgttgaaatcgggtcaattcgatccctcgtcacctatgaatggtaatgggagaggatttaagttcgattcgcgtgatgcggaagatatttttgcggaattttttggtggatcggatgggtatagtaggagtcctactggtggtactgtacggattaggaagccggcgccggtggagaacaagttgccatgtagcttggaggaattgtacaagggttctaagaggaagatgaagatttcaaggattgttcttgatgtcactggtaagcctacaacaattgaagaggtcttggcaatacacattaaacctggttggaagaaaggcacaaaaatcacttttccagagaaagggaaccatgaacctggagctgcacctggtgatcttatttttgtaatcgatgaaaagccacatgatgtcttcaagagagatggaaatgatctagtgatcaatcagaaaatctcattagtagatgctctctctgggaagattatcaacttggctactttggatggaagggaactcacgataccaatcacagatgttgttaagccaggacatgagcagataatcgcagatgaaggaatgccaatatcaaaagaacccgggaagaaaggaaatttgaggatcaagtttgaggttaagttcccgtcaaggcttagttcagatcagaaattggatatcagaagagtgctgggcaggactgttgactaatccagtgcttaacttaaggtatcactaacactgactgattgtggaagtgcaatggaatgtaaatacttggaaagtttatgctagacaggagaattgtgcatagtaggattattgttttgacgatgcctctcaaagtttagttttatttagcgccataaaaggagcactctagcagctatgccagctgcctatgtaattctcatgttggatattaaacaccttaggtagtttactaagttttaatgtttgatttggaggtagatgctatacttcaagtaattggagtcgggaatagcttgctatcttgttctggttgtgaatttcaactgtgggctgtggtagactggtaatgaataaaaaaaaaaaaaaaaaaaaaaaaaaaaaaaaaaa

SEQ ID NO: 2 - DNA, 918 bp, Nicotiana tabacum
  gene (1)..(918): NtCPIP2a
atgggacttgattactataacgtactaaaagtatctcggaatgcaagtgaagaagatttaaagagatcgtacaagcgattagctatgaaatggcatccagataagaacagtcagaacaaaaaagaagcagaagcaaaattcaagcagatttctgaagcgtatgatgtgcttagtgatcctaagaagcgtcagatctatgacgtgtacggtgatgatgcattgaaatccggtcaatttgcctcggcgtcgccgactagtgctggtagtaacgccagagggtttaggttcaatacgcgtgatgcggaggctatttttgctgagtttttcggtggatcgggtagtaattccggtgccggagtcggtcggaaggcggcgccggtggagaataaactgccgtgtagcttggaggagctttacaaaggttctagaagaaaaatgaagatctcacggattcttctggatgactctggtaagcctacaactgttgaagaggtcttagcgatacacatcaagcctggttggaagaaaggcacaaaaatcactttcccagagaaaggaaactatgagcctggagctactcctggtgatcttatttttgtgatagatgaaaagccgcatgctgtcttcaagagggatggaaatgatctggagatcaatcagaaaatttctttactagatgctcttactgggaaaactataagcttgatcactttggatggacgggaactcacaataccaatcacagatattgttaaaccaggacatgagcatataatcccaaatgaaggaatgccaatatcaaaggaacgtggcaagaaaggaaatttgaagatcaagtttgacattaaattcccatcaaggctaagtgcagatcagaaatctgatatcaggagggtactgtgcaggagcgctgactaa

SEQ ID NO: 3 - DNA, 918 bp, Nicotiana tabacum
  gene (1)..(918): NtCPIP2b
atgggacttgattactatgacgtactgaaagtatctcggaatgcaagtgaagaagatttaaagagatcgtataagcgattagcgatgaaatggcatccagataagaacagtcagaacaaaaaggaagctgaagcgaaattcaagcagatttctgaagcgtatgatgtgcttagtgatcctcagaagcgtcagatctatgacgtgtacggtgatgatgcattgaaatccggtcaatttgcttcggcgtcgccgactagtgctggtagtaacggcagaggatttaggttcaatacgcgtgacgcggaggctattttcgctgagtttttcggtggatcggatagtaattccgctgccggagtaggtcggaaggcggcaccggtggagaataaactgccgtgtagcttggaggagctttacaaaggatctagaaggaaaatgaagatctcacggattcttcttgatgactctggtaagcctacaactgttgaagaggtcctagcgatacacatcaaaccaggttggaagaaaggcacaaaaatcactttcccagagaaagggaactatgaacctggagctactcctggtgatcttatttttgtgatagatgaaaagccacatgctgtcttcaagagggatggaaatgatctagtgatcaatcagaaaatatctttactagatgctcttactgggaaaactataagcttgatcactttggatggacgggaactcacaataccaatcacagatgttgttaaaccaggacatgagcatataatcccaaatgaaggaatgccaatatcaaaggaacgtggcaagaaaggaaatttgaagatcaagtttgacattaaattcccatctaggctaagtgcagatcagaaatctgatatcaggagggtactgtgcaggaactctgactaa

SEQ ID NO: 4 - protein, 306 aa, Nicotiana tabacum
  PEPTIDE (1)..(306): NtCPIP1
MGVDYYKILKVSRNASEEDLKKSYKRLAMKWHPDKNSEKEAEAKFKQISEAYDVLSDPQKRQIYDIYGDEALKSGQFDPSSPMNGNGRGFKFDSRDAEDIFAEFFGGSDGYSRSPTGGTVRIRKPAPVENKLPCSLEELYKGSKRKMKISRIVLDVTGKPTTIEEVLAIHIKPGWKKGTKITFPEKGNHEPGAAPGDLIFVIDEKPHDVFKRDGNDLVINQKISLVDALSGKIINLATLDGRELTIPITDVVKPGHEQIIADEGMPISKEPGKKGNLRIKFEVKFPSRLSSDQKLDIRRVLGRTVD

SEQ ID NO: 5 - protein, 305 aa, Nicotiana tabacum
  PEPTIDE (1)..(305): NtCPIP2a
MGLDYYNVLKVSRNASEEDLKRSYKRLAMKWHPDKNSQNKKEAEAKFKQISEAYDVLSDPQKRQIYDVYGDDALKSGQFASASPTSAGSNARGFRFNTRDAEAIFAEFFGGSGSNSGAGVGRKAAPVENKLPCSLEELYKGSRRKMKISRILLDDSGKPTTVEEVLAIHIKPGWKKGTKITFPEKGNYEPGATPGDLIFVIDEKPHAVFKRDGNDLEINQKISLLDALTGKTISLITLDGRELTIPITDIVKPGHEHIIPNEGMPISKERGKKGNLKIKFDIKFPSRLSADQKSDIRRVLCRSAD

SEQ ID NO: 6 - protein, 305 aa, Nicotiana tabacum
  PEPTIDE (1)..(305): NtCPIP2b
MGLDYYDVLKVSRNASEEDLKRSYKRLAMKWHPDKNSQNKKEAEAKFKQISEAYDVLSDPQKRQIYDVYGDDALKSGQFASASPTSAGSNGRGFRFNTRDAEAIFAEFFGGSDSNSAAGVGRKAAPVENKLPCSLEELYKGSRRKMKISRILLDDSGKPTTVEEVLAIHIKPGWKKGTKITFPEKGNYEPGATPGDLIFVIDEKPHAVFKRDGNDLVINQKISLLDALTGKTISLITLDGRELTIPITDVVKPGHEHIIPNEGMPISKERGKKGNLKIKFDIKFPSRLSADQKSDIRRVLCRNSD

SEQ ID NO: 7 - protein, 66 aa, Nicotiana tabacum
  DOMAIN (1)..(66): consensus sequence of the DnaJ-like domain
DYYEILGVPRDASDEEIKKAYRKLALKYHPDKNPGDPEAEEKFKEINEAYEVLSDPEKRAIYDQYG

SEQ ID NO: 8 - DNA, 801 bp, CP cDNA from potato virus Y (PVY)
gcaaatgacacaattgatgcaggaggaagctctaagaaagatgcaaaccaagagcaaagtagcattcaaccaaatcccaacaagagaaaggaaaaggacgtgaatgttggaacatctggaactcacactgtgccacgaattaaagctatcacgtccaaaatgagaatgcccaagagtaagggtgcaactgtactaaatttagaacatttactcgagtatgctccacagcaaattgacatctcaaatactcgagcaactcaatcacagtttgatacatggtatgaagcagtacaacttgcatacgacataggagaaactgaaatgccaactgtgatgaatgggcttatggtttggtgcattgaaaatggaacctcgccaaatatcaatggagtttgggttatgatggatggagatgaacaaatcgaatacccactaaaaccaatcgttgagaatgcaaaaccaacacttaggcaaatcatggcacatttctcagatgttgcagaagcgtacatagaaatgcgcaacaaaaaggaaccatacatgccacgatatggtttagttcgtaatctgcgcgatggaagtttggctcgctatgcttttgacttttatgaagtcacatcacgaacaccagtgagggctagagaggcacacattcaaatgaaggccgcagctttaaaatcagctcaatctcgacttttcggattggatggtggcatcagtacacaagaggaaaacacagagaggcacaccaccgaggatgtttctccaagtatgcatactctacttggagtcaagaacatg

SEQ ID NO: 9 - protein, 267 aa, CP from potato virus Y (PVY)
ANDTIDAGGSSKKDANQEQSSIQPNPNKRKEKDVNVGTSGTHTVPRIKAITSKMRMPKSKGATVLNLEHLLEYAPQQIDISNTRATQSQFDTWYEAVQLAYDIGETEMPTVMNGLMVWCIENGTSPNINGVWVMMDGDEQIEYPLKPIVENAKPTLRQIMAHFSDVAEAYIEMRNKKEPYMPRYGLVRNLRDGSLARYAFDFYEVTSRTPVRAREAHIQMKAAALKSAQSRLFGLDGGISTQEENTERHTTEDVSPSMHTLLGVKNM

SEQ ID NO: 10 - DNA, 918 bp, Nicotiana tabacum
atgggacttgattactataacgtactaaaagtatctcggaatgcaagtgaagaagatttaaagagatcgtacaagcgattagctatgaaatggcatccagataagaacagtcagaacaaaaaagaagcagaagcaaaattcaagcagatttctgaagcgtatgatgtgcttagtgatcctcagaagcgtcagatctatgacgtgtacggtgatgatgcattgaaatccggtcaatttgcctcggcgtcgccgactagtgctggtagtaacgccagagggtttaggttcaatacgcgtgatgcggaggctatttttgctgagtttttcggtggatcgggtagtaattccggtgccggagtcggtcggaaggcggcgccggtggagaataaactgccgtgtagcttggaggagctttacaaaggttctagaagaaaaatgaagatctcacggattcttctggatgactctggtaagcctacaactgttgaagaggtcttagcgatacacatcaagcctggttggaagaaaggcacaaaaatcactttcccagagaaaggaaactatgagcctggagctactcctggtgatcttatttttgtgatagatgaaaagccgcatgctgtcttcaagagggatggaaatgatctggagatcaatcagaaaatttctttactagatgctcttactgggaaaactataagcttgatcactttggatggacgggaactcacaataccaatcacagatattgttaaaccaggacatgagcatataatcccaaatgaaggaatgccaatatcaaaggaacgtggcaagaaaggaaatttgaagatcaagtttgacattaaattcccatcaaggctaagtgcagatcagaaatctgatatcaggagggtactgtgcaggagcgctgactaa

(19) United States
(12) Patent Application Publication
(10) Pub. No.: US 2005/0251879 A1
(43) Pub. Date: Nov. 10, 2005

(54) Method for the production of transgenic plants with increased virus resistance by silencing vegetable DnaJ-like proteins

(76) Inventors: Daniel Hofius, Copenhagen (DK); Frederik Bornke, Quedlinburg (DE); Uwe Sonnewald, Quedlinburg (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/039,722

(22) Filed: Jan. 19, 2005

Related U.S. Application Data
(63) Continuation of application No. PCT/EP03/07945, filed on Jul. 21, 2003.

(30) Foreign Application Priority Data
Jul. 19, 2002 (DE) 102 32 978.8

(51) Int. Cl. A01H 1/00; C12N 15/82

(52) U.S. Cl. 800/280; 435/468

(57) ABSTRACT

The invention relates to plants and plant cells which have a transient or permanent virus resistance as a result of modulation of the gene expression and/or binding behavior of vegetable DnaJ-like proteins. The invention also relates to methods for the production of transgenic plants with increased virus resistance, wherein the expression of vegetable DnaJ-like proteins which interact with viral components is substantially prevented by silencing the DnaJ-like proteins. The invention further relates to methods for the production of transgenic plants with increased virus resistance, wherein the interaction of viral components with vegetable DnaJ-like proteins is substantially prevented by expression of dominant-negative mutants of the DnaJ-like proteins, by antibodies against DnaJ-like proteins or by specific inhibitors.

### RELATED APPLICATIONS

The present application is a continuation of and claims priority under 35 U.S.C. §120 to PCT Application Serial No. PCT/EP2003/007945, filed Jul. 21, 2003 by Hofius et al., entitled “METHOD FOR THE PRODUCTION OF TRANSGENIC PLANTS WITH INCREASED VIRUS RESISTANCE BY SILENCING VEGETABLE DNAJ-LIKE PROTEINS,” and which claims priority to German Patent Application No. 102 32 978.8, filed Jul. 19, 2002, both of which are herein incorporated by reference in their entirety.

### BACKGROUND OF THE INVENTION

1. Field of the Invention

The present invention relates to plants and plant cells which have a transient or permanent virus resistance due to a modulation of the gene expression and/or the binding characteristics of plant DnaJ-like proteins, as well as methods for the production of such plants and plant cells.

2. Description of the Related Art

There are numerous plant viruses which infest important agricultural plants and lead to significant crop failures each year. Accordingly, for a long time there has been a requirement for methods which make it possible to produce plants with increased virus resistance.

In order to produce transgenic plants with increased virus resistance, different strategies can be chosen which generally depend upon the virus type and also upon the infection mechanism used by the virus.

The most widely adopted strategy is the so-called “pathogen mediated resistance” strategy (summarized in Baulcombe 1996, Plant Cell, 8:1833-1844). With this strategy, nucleic acid sequences, which either correspond to viral sequences or are complementary to viral sequences, are transferred to plant cells. Depending upon whether these transferred nucleic acid sequences are transcribed and translated or just transcribed, one can distinguish between the protein-based or RNA-based pathogen mediated resistance.

With protein-based pathogen mediated resistance, viral capsid proteins or so-called movement proteins which are responsible for the transport of the virus between and within the plant cells, and therefore for the systemic infection of the plants, are generally expressed in the transgenic plant cells. The observed virus resistance is probably based upon the fact that by the expression of the viral components in the case of viral capsid proteins, the assembly of functional virus particles, and in the case of viral movement proteins, the propagation of the virus within the plant, is outcompeted.

The RNA-based pathogen mediated resistance is based upon the transcription of viral sequences. These viral sequences can encode viral products, but need not do so. The basis for the RNA-based pathogen mediated resistance is to be seen in a process which is called “post-transcriptional gene silencing”(PTGS) and is based upon the appearance of double-stranded RNA molecules in the plant cell. Accordingly, the possibilities for establishing an RNA-based pathogen mediated resistance are numerous. For example, viral sequences can be used which are found in a sense or antisense orientation. When transcribing viral sequences with antisense orientation, double-stranded RNA molecules inevitably occur during viral infections. But also with the transcription of viral sequences in sense orientation which have pronounced complementarity within a transcript, double stranded RNA regions can occur. Moreover, double-stranded RNA molecules inevitably occur with many viral infections because many plant viruses, such as potyviruses, have an RNA genome with (+) polarity and are replicated via a double-stranded RNA intermediate.

The occurrence of double-stranded RNA molecules within a plant cell induces the PTGS system which brings about specific degradation of the corresponding viral sequences. This mechanism, also termed “silencing” and known as “RNA interference” (RNAi) in C. elegans, occurs naturally with infection of plants with plant viruses. However, it is often not induced strongly enough to stave off the virus infection. Transgenic plants which are equipped with an RNA with homology, e.g. to a viral protein, generally transcribe this RNA to a sufficient extent so as to induce the PTGS system, and so also virus resistance. Because of this, the plant PTGS system can also be induced specifically by introducing viral double-stranded RNA molecules. In this way, stable transgenic plants with the required virus resistance can be produced (Waterhouse et al., 2001, Nature, 411, 834-842).

The disadvantage of using pathogen mediated resistance mechanisms in order to produce transgenic plants with increased virus resistance is that, due to the necessary complementarity of the transgene RNA to the viral RNA, the plants are only resistant to a very specific virus. They are only equipped, therefore, with a limited resistance spectrum. In addition, viruses have developed mechanisms by means of which the PTGS system induced by viral sequences can possibly be repressed. With potyviruses, e.g., the “helper component proteinase” (Hc-Pro) is involved (Kaschau et al., 1998, Cell: 461-470).

Other strategies for the production of plants with increased virus resistance are based upon the fact that proteases, which are required by viruses in the course of their replication cycle, are selectively inhibited. The activity of cysteine proteinases could thus be inhibited by overexpression of a cysteine proteinase inhibitor from rice, and plants with increased resistance to potyviral infections could be produced (Gutierrez-Campos et al., 1999, Nat. Biotechn. 17: 1223-1226).

Other strategies for the production of transgenic plants with increased virus resistance are based upon the transfer of generally antiviral acting systems to plant cells. The expression of proteins which affect the different components of the viral replication cycle, also plays a role. An example of this are transgenic plants which express the so-called “pokeweed antiviral protein” (PAP), a ribosome inactivating protein, and which, for reasons which are not understood, exhibit resistance to a broad spectrum of viruses. However, transgenic plants which express the PAP protein show significantly reduced growth dependent upon the expression level (Lodge et al., 1993, PNAS, 90: 7089-7093, Turner et al., 1997, PNAS, 94: 3866-3871).

Another example for plants which are equipped with a generally antiviral acting system, are plants into which the interferon-inducible antiviral 2,5-oligoadenylate system is introduced from animal organisms. The plants constitutively express a 2,5-oligoadenylate synthetase and a 2,5-oligoadenylate-dependent ribonuclease L, with can be activated by double-stranded RNA (Mitra et al., 1996, PNAS, 93: 6780-6785). Moreover, there are further attempts to increase the plants' virus resistance which have other mechanistic bases which are often still not understood in detail. One possibility to increase the plants' virus resistance is the induction of unspecific plant defense systems, as achieved by the so-called “systemic acquired resistance”, e.g. by the constitutive overexpression of salicylic acid (Verberne et al., 2000, Nat. Biotechn., 18: 779-783).

In addition, the transfer of specific virus resistance genes to the respective plants is appropriate for the production of plants with increased virus resistance (Bendahmane 1999 et al., Plant Cell, 11: 781-791). The disadvantage of this type of transgenic plant, however, is that it is only resistant to a narrow spectrum of different virus groups or strains.

Because of the great significance of virus-resistant plants for agriculture, there is in general a great need for further methods which allow the production of transgenic plants with increased virus resistance. In particular, there is a need for methods which make it possible to produce transgenic plants with increased virus resistance and which are resistant to a broad spectrum of virus groups and strains.

### SUMMARY OF THE INVENTION

The objective of the present invention is to provide a method alternative to the methods known in the prior art, which makes it possible to produce plants with increased virus resistance. In particular, it is an objective of the present invention to provide a method which allows the production of plants which exhibit resistance to a broad spectrum of virus groups and strains. Another objective which underlies the invention is to provide a method for the production of plants with increased virus resistance which is generally applicable and easy to put into practice.

The objective is achieved by the present invention in that a method for the production of transgenic plants with increased virus resistance is provided, characterized in that the expression of plant DnaJ-like proteins which interact with viral components is essentially suppressed. The objective is also achieved by a method for the production of transgenic plants whereby the interaction of viral components with plant DnaJ-like proteins which interact with viral components, is substantially prevented by the expression of dominant negative mutants of DnaJ-like proteins or antibodies to DnaJ-like proteins.

Accordingly, the invention relates to methods for the production of plants and plant cells with increased virus resistance, which is characterized in that the interaction of viral components with DnaJ-like proteins is essentially suppressed and substantially prevented, respectively, by reducing the expression of plant DnaJ-like proteins. Moreover, the invention relates to a method for the production of plants and plant cells with increased virus resistance, characterized in that the interaction of viral components with plant DnaJ-like proteins is essentially blocked by dominant negative mutants of the DnaJ-like proteins, by antibodies against DnaJ-like proteins or by specific inhibitors.

Moreover, the present invention relates to a method for the production of transgenic plants and plant cells with increased virus resistance, characterized in that the interaction of viral components with plant DnaJ-like protein is essentially suppressed and substantially prevented, respectively, by silencing the plant DnaJ-like proteins. Moreover, the invention relates to a method for the production of transgenic plants and plant cells with increased virus resistance, characterized in that the interaction of viral components with DnaJ-like proteins is essentially blocked by dominant negative mutants of the DnaJ-like proteins, by antibodies against DnaJ-like proteins or by specific inhibitors.

The instant invention additionally relates to plants and plant cells with increased virus resistance, characterized in that the interaction of viral components with DnaJ-like proteins is essentially suppressed and substantially prevented by reducing the expression of plant DnaJ-like proteins.

The present invention additionally relates to a method for the production of transgenic plants with increased virus resistance, characterized in that the expression of plant DnaJ-like proteins, which interact with viral components, is substantially prevented. The invention also provides such methods characterized in that the expression of plant DnaJ-like proteins is substantially prevented by means of transfer of nucleic acid molecules comprising sequences, which are identical, homologous or complementary to the sequences encoding plant DnaJ-like proteins or fragments thereof, to plant cells. The methods of the invention additionally relate to methods characterized in that the transferred nucleic acid molecules comprise sequences, which are identical, homologous or complementary to the sequences encoding the J-domain of plant DnaJ-like proteins or fragments thereof, or that are characterized in that the transferred nucleic acid molecules comprise sequences, which are identical, homologous or complementary to the sequences encoding the protein specific regions of plant DnaJ-like proteins, which do not coincide with the J-domain, or fragments thereof.

The methods of the invention may also be characterized in that the fragment of the transferred nucleic acid sequences, which is identical, homologous or complementary to the sequences encoding plant DnaJ-like proteins or fragments thereof, comprises 20 to 1000 nucleotides, 20 to 750 nucleotides, preferably 20 to 500 nucleotides, also preferably 20 to 250 nucleotides, especially preferably 20 to 150 nucleotides, particularly preferably 20 to 100 nucleotides, and most preferably about 20-50 nucleotides. The methods of the invention may be further characterized in that a fragment of the transferred nucleic acid sequences may be at least 50% homologous to the sequences encoding plant DnaJ-like proteins or fragments thereof, preferably at least 60%, also preferably at least 70%, especially preferably at least 80%, particularly preferably at least 90%, and most preferably at least 95%. The methods may also be characterized in that a fragment of the transferred nucleic acid sequences is at least 50% complementary to the sequences encoding plant DnaJ-like proteins or fragments thereof, preferably at least 60%, also preferably at least 70%, especially preferably at least 80%, particularly preferably at least 90%, and most preferably at least 95%.

In some embodiments, the methods of the invention comprise: producing a vector which comprises the following sequence elements in 5′-3′ orientation: a promoter being functional in plants; operatively linked thereto the identical or homologous antisense sequence of the sequence encoding the plant DnaJ-like protein or fragments thereof, wherein the sequence exhibits 3′exon sequences at its 3′end, being recognizable by the spliceosome; an intron; the identical or homologous sense sequence of the sequence encoding the plant DnaJ-like protein or fragments thereof, wherein the sequence exhibits 5′exon sequences at its 5′end, being recognizable by the spliceosome; and a termination sequence; followed by transferring the vector to plant cells and, optionally, integration into the plant genome.

In other embodiments, the methods of the invention comprise producing a vector which comprises the following sequence elements in 5′-3′ orientation: a promoter being functional in plants; operatively linked thereto the identical or homologous antisense sequence of the sequence encoding a plant DnaJ-like protein or fragments thereof; a termination sequence; followed by transferring the vector to plant cells and, optionally, integration into the plant genome.

Still other embodiments provide methods comprising, producing a vector which comprises the following sequence elements in 5′-3′ orientation: a promoter being functional in plants; operatively linked thereto the identical or homologous sense sequence of the sequence encoding a plant DnaJ-like protein or fragments thereof, wherein the sequence has self-complementary regions; a termination sequence; followed by transferring the vector to plant cells and, optionally, integration into the plant genome.

Yet other embodiments provide methods comprising, producing a vector which comprises the following sequence elements in 5′-3′ orientation: a promoter being functional in plants; operatively linked thereto a DNA sequence, which is complementary to the sequence encoding the mRNA of the plant DnaJ-like protein or fragments thereof; a DNA sequence encoding ribonuclease P; a termination sequence, followed by transferring the vector to plant cells and, optionally, integration into the plant genome.

Still further embodiments provide methods comprising producing a vector which comprises the following sequence elements in 5′-3′ orientation: a promoter being functional in plants; a DNA sequence which is identical or homologous to the sequence encoding the 5′end of the plant DnaJ-like protein; a DNA sequence encoding a resistance gene; a DNA sequence which is identical or homologous to the sequence encoding the 3′end of the plant DnaJ-like protein; a termination sequence; followed by transferring the vector to plant cells and integration into the plant genome.

Additional embodiments relate to methods comprising producing a vector which comprises the following sequence elements in 5′-3′ orientation: a promoter being functional in plants; operatively linked thereto a DNA sequence encoding a ribozyme which specifically recognizes the mRNA of plant DnaJ-like proteins; a termination sequence; followed by transferring the vector to plant cells and, optionally, integration into the plant genome.

In yet additional embodiments, methods are provided which comprise producing a vector which comprises the following elements in 5′-3′ orientation: a promoter being functional in plants; operatively linked thereto a DNA sequence encoding a dominant-negative mutant of a plant DnaJ-like protein, which interacts with viral components, or a recombinant antibody, which is specific for plant DnaJ-like proteins; a termination signal; followed by transferring the vector to plant cells and, optionally, integration into the plant genome.

The above methods may be further characterized in that: the vector contains further regulatory and functional sequences in addition to promoters; the regulatory sequences are enhancers, replication signals, selection markers and/or sequences allowing for a propagation of the vectors in bacteria and/or a replication in plant cells; the vectors are plasmids, cosmids and/or recombinant viruses; the vectors are pBR322, pUC vectors, M13 mp vectors or vectors derived from the Agrobacterium Ti or Ri plasmid; the promoters are constitutive promoters, preferably the ³⁵S, the actin or the ubiquitin promoter, tissue-specific promoters, preferably the phosphoenolpyruvate carboxylase or the fructose-1,6-bisphosphatase promoter, developmental-specific, light-, wound- or pathogen-induced promoters, preferably virus-induced promoters; the vector is transferred to the plants by means of transformation, transfection, injection, biolistic methods and/or electroporation; the plant DnaJ-like proteins contain a J-domain, which is at least 40% homologous to the amino acid sequence SEQ ID NO. 7, preferably at least 50%, especially preferably at least 60%, particularly preferably at least 70%, also particularly preferably at least 80%, and most preferably up to 90%; the plant DnaJ-like proteins are encoded by the sequences SEQ ID NO. 1, 2 or 3; or in that the plant DnaJ-like proteins are at least 40% homologous to the amino acid sequence SEQ ID NO. 4, 5 or 6, preferably at least 50%, especially preferably at least 60%, particularly preferably at least 70%, also particularly preferably at least 80%, and most preferably at least 90%.

The above methods may additionally by characterized in that: the transgenic plants exhibit increased resistance to Poty, Carla, Hordei, Potex, Bunyai, Clostero, Cucumo, Diantho, Tobamo, Gemini, Lutero, Rymo, Tobra, Furo, Como, Nepo, Bromo or Tospo viruses; the plants exhibit increased resistance to PVY, to TEV and/or to TSWV; the transgenic plants are monocotyledonous plants, preferably plants belonging to the genera Avena (oat), Triticum (wheat), Secale (rye), Hordeum (barley), Oryza (rice), Panicum, Pennisetum, Setaria, Sorghum (millet), Zea (maize) and the like; or in that the transgenic plants are dicotyledonous plants, preferably cotton, leguminous plants such as legumes and especially alfalfa, soy bean, rape, tomato, sugar beet, potato, ornamental plants, tobacco as well as trees.

The present invention also provides transgenic plant cells or plants with increased virus resistance, which may be produced by any of the above methods. In addition, such plant cells or plants may be further characterized in that: the expression of plant DnaJ-like proteins, which interact with virus components, is substantially prevented; the plant DnaJ-like proteins are encoded by SEQ ID NO. 1, 2 or 3; the plant DnaJ-like proteins are at least 30% homologous to the amino acid sequence SEQ ID NO. 4, 5 or 6, preferably at least 40%, especially preferably at least 50%, also especially preferably at least 60%, particularly preferably at least 70%, also particularly preferably at least 80%, and most preferably at least 90%; dominant-negative mutants of plant DnaJ-like proteins, which interact with virus components, or antibodies being specific for DnaJ-like proteins are expressed in the plant cells or the plant; dominant-negative mutants of DnaJ-like proteins and preferably of plant DnaJ-like proteins, which interact with virus components, are expressed in the plant cells or the plant; the plant DnaJ-like proteins are encoded by SEQ ID NO. 1, 2 or 3; the plant DnaJ-like proteins are at least 40% homologous to the amino acid sequence SEQ ID NO. 4, 5 or 6, preferably at least 50%, especially preferably at least 60%, particularly preferably at least 70%, also particularly preferably at least 80%, and most preferably at least 90%; proteins are expressed as dominant-negative mutants of the DnaJ-like proteins and preferably of the plant DnaJ-like proteins, in which the J-domain is deleted; or in that N-terminal deletion mutants of the plant DnaJ-like proteins, which are encoded by SEQ ID NO. 1, 2 or 3, are expressed as dominant-negative mutants, in which the J-domain is deleted; or the J-domain is overexpressed.

The invention also provides virus resistant plant cells or plants, characterized in that: they contain mutations in the coding and/or regulatory sequences of the genes for plant DnaJ-like proteins, which cause a modulation of the expression of these proteins and/or their binding behavior to viral proteins and/or endogenous cellular binding partners; a dominant-negative mutant of plant DnaJ-like proteins is expressed, which is not able to interact with viral components and/or the cellular interaction partners of plant DnaJ-like proteins; or in that they can be produced by the “TILLING” method.

These and other objects and features of the invention will become more fully apparent when the following detailed description is read in conjunction with the accompanying drawings.

### BRIEF DESCRIPTION OF THE DRAWINGS

FIG. 1: a) Alignment of the proteins NtCPIP1, NtCPIP2a, NtCPIP2b and NtDnaJ_M541 which interacts with the TSWV movement protein (NSm) (Soellick et al., 2000). b) Homology of the protein sequences in percent of identical amino acids, determined according to the Clustal method in the DNAStar package, DNASTAR Inc., Madison, Wis. c) Consensus sequence of the DnaJ domain. Nter and Cter stand for N terminus and C terminus and indicate the orientation of the sequence.

FIG. 2a: NtCPIP1 cDNA (SEQ ID NO:1) and derived amino acid sequence (SEQ ID NO:4) from Nicotiana tabacum. The amino acids identified in the yeast 2 hybrid system and interacting with PVY CP are highlighted. The eponymous J domain is underlined. The lys-rich domains have grey background.

FIG. 2b: NtCPIP2a cDNA (SEQ ID NO: 2) with derived amino acid sequence (SEQ ID NO: 5).

FIG. 2c: NtCPIP2b cDNA (SEQ ID NO: 3) with derived amino acid sequence (SEQ ID NO: 6).

FIG. 3: PVY CP cDNA with derived amino acid sequence.

FIG. 4: Specific interaction between the Potato virus Y capsid protein (PVY CP) and the DnaJ-like protein NtCPIP1 from Nicotana tabacum in the yeast 2 hybrid system. The PVY CP (pGBT9-PVY CP) fused with the Gal4 binding domain (BD) only shows lacZ activity when co-expressed with NtCPIP1 (pAD-CPIP1) fused with the Gal4 activation domain, whereas in control reactions, NtCPIP1 brings about no activity with the binding domain alone (pGBT1) or with the unrelated protein SNF1 (pBD-SNF1). Accordingly, the co-expression of PVY CP and SNF4 (pAD-SNF4)— the binding partner of SNF1—shows no activity, whereas SNF1 and SNF4 induce clear lacZ activity in the positive control.

FIG. 5: LacZ filterlift assay for the analysis of the protein-protein interactions of NtCPIP1, NtCPIP2a, NtCPIP2b and NtDnaJ-M54 (Soellick et al., 2000, vide supra) with potyviral CPs (PVY, TEV, TVMV, TuMV) and the TSWV NSm movement protein (Soellick et al., vide supra) in the yeast 2 hybrid system.

FIG. 6: PVY CP deletion mutants and different point mutants were co-transformed respectively with NtCPIP1 in Y190 cells and qualitatively analyzed with regard to B galactosidase activity.

FIG. 7: Schematic representation of the pUC RNAi vector.

FIG. 8: Schematic representation of the pBinNtCPIP 1-RNAi vector.

FIG. 9: a) Northern blot analysis of NtCPIP1-specific transcripts. Total RNA was isolated either from WT plants or from transgenic plants, and hybridized with NtCPIP1 cDNA. b) and c) Analysis of the NtCPIP1 suppression by means of RT-PCR. In B) the PCR products for NtCPIP1 and actin from the PCR cycles 30 and 25 are shown.

FIG. 10: a) PVY virus titer in locally infected leaves from NtCPIP1—RNAi transgenic lines. PVY capsid protein levels were determined 4 days post infection in the locally infected leaves from the lines NtCPIP1—RNAi-2 and -3 and in control plants, as described in Herbers et al. 1996. The results are shown in % of the wild type (control) level from two independent experiments. The values represent the average value of the indicated number of plants (n) ±standard error. b) PVY virus titer in locally infected and systemic leaves from NtCPIP1—RNAi transgenic lines. PVY capsid protein levels were determined 6 days post infection in the locally and systemically infected leaves from the lines NtCPIP1-RNAi-2 and -3 as well as in control plants, as described in Herbers et al. 1996. The results are given in % of the wild type (control) level. The values represent the average value of the indicated number of plants (n) ±standard error.

FIG. 11: Effect of PVY infections in NtCPIP1 silenced transgenic plants. a) The virus titer was determined four days post infection in local (inoculated) leaves from the lines NtCPIP1—RNAi-2, -3, -69 and -79 and WT plants and transgenic control plants ME-4. b) The virus titer was determined four days post infection in local (inoculated) or systemic leaves (4-5 leaves above the local (inoculated) leaves) from the lines NtCPIP1-RNAi-2, -3, -69 and -79 and WT plants or transgenic control plants ME-4. The plants had developed six leaves prior to infection.

FIG. 12: Expression of dominant negative mutants of NtCPIP1 and 2a in tobacco plants. a) Interaction of N terminal deleted fragments of NtCPIP 1 and 2a with PVY CP in the yeast “two hybrid” system. b) Schematic representation of the binary constructs used for expression. c) Northern blot analysis of the NtCPIP1Δ65N and NtCPIP2aΔ66N specific transcripts. Total RNA was isolated from control or transgenic plants and hybridized with NtCPIP1Δ65N or NtCPIP2aΔ66N cDNA. d) PDY titer in systemic leaves (5-6 leaves above the inoculated leaves) 6 days post infection. e) Development of virus-induced symptoms in control and transgenic plants 13 days post infection.

FIG. 13: Another DNA sequence for a DnaJ-like protein from Nicotiana tabacum (SEQ ID NO: 10).

### DETAILED DESCRIPTION OF THE INVENTION

Whereas with the pathogen mediated resistance the function of a viral component is specifically prevented, and with other strategies for the generation of virus-resistant plants, such as the expression of PAP protein or the transfer of the 2.5 oligoadenylate system from animal to plant cells, antiviral acting systems are generally introduced into the plants, an essentially different strategy is adopted according to the present invention.

It was surprising to find that by the suppression or silencing of plant host genes, of which the protein products, by interacting with virus components, are of great significance to the replication, assembly and spreading of the virus, plants can be produced which have increased virus resistance, without these plants displaying phenotype interference to any significant extent. This finding was particularly unexpected in view of the great significance of DnaJ-like proteins for the functional integrity of cellular systems.

The DnaJ protein or DnaJ-like proteins (also called HSP40 proteins or HSP40-like proteins) are important representatives of the chaperone system of cells. Chaperones are proteins or protein complexes which are involved in a variety of cellular processes. These include, amongst others, the active folding of proteins, the prevention of protein aggregation, the break-up of protein aggregates, the correct folding of misfolded proteins, the generation and break-up of cytoskeletal structures and protein targeting into different cellular compartments (Harti et al., 2002, Science, 295, 1852-8 and 1996, Nature, 381, 571-9).

DnaJ-like proteins are characterized by the presence of a so-called J domain. They interact with other chaperones such as DnaK-like proteins (also called Hsp70 or Hsc70 proteins), the ATPase activity of which they can stimulate. DnaK proteins are equipped with an ATP- and a substrate-binding domain. In its ATP-bound state DnaK only has a small affinity for its substrates.

DnaJ proteins bind via the J domain to DnaK proteins and stimulate the latter's ATPase activity, the result of which is that the DnaK proteins can bind a greater substrate spectrum because of the activation by the DnaJ proteins (Kelley, 1999, Current Biology, 9: 305-308 and Kelley, 1998, TIBS, 23: 222-227). In this way, DnaJ proteins, amongst other things, act as adaptors for DnaK protein substrates and extend the substrate specificity of the same. Both DnaJ and DnaK proteins are equipped with numerous homologues in various species. There is increasing evidence to show that specific members of the DnaK family only interact with specific members of the DnaJ family (Kelley, 1999, Current Biology, 9: 305-308). The presence of a J domain, by means of which these proteins bind to their DnaK partners, is crucial for the function of the DnaJ proteins.

Viruses, which attack plant or animal cells, also take advantage of the capability of J domains to recruit other chaperones. Some viral proteins bind, e.g., directly to Hsp70 proteins. Other viral proteins, such as the “large T antigen” are equipped with J domains. It is thought that these viral proteins use the cellular chaperone system in order, either to disassemble or assemble viral protein complexes, or to break-up cellular complexes which prevent the spreading of viruses within the cell (Sullivan et al., 2001, Virology, 287: 1-8, Kelley, 1998, TIBS, 23: 222-227).

For plant closteroviruses it has been shown, e.g., that they contain their own Hsp70-like protein which is essential for the assembly and spreading of the viruses (Alzhanova et al., 2001, EMBO J., 20: 6997-7007). Moreover, it was possible to show that the movement protein NSm of the tomato spotted wilt virus (TSWV) interacts with DnaJ-like proteins from Arabidopsis thaliana and Lycopersicon esculentum (Von Bargen et al., 2001, Plant Physiol. Biochem., 39: 1083-1093 and Soellick et al., 2000, PNAS, 97: 2373-2378). Within the framework of the present invention, it is also shown that the capsid protein CP of the potyviruses potato virus Y (PVY) and tobacco etch virus (TEV) also interacts with a DnaJ-like protein (see examples).

Therefore, it can be assumed that plant viruses make use of the functions of the cellular chaperone system during the infection and also use, amongst other things, DnaJ-like proteins. In this way, the DnaJ-like proteins theoretically provide a point of attack for antiviral strategies. Because of their outstanding significance as chaperones for various cellular functions in the non-infected host, it could, however, not be concluded that the suppression of DnaJ-like proteins would lead to viable plants with a phenotype which has not essentially been altered.

With the present invention, it was possible to show that suppression of the expression of DnaJ-like proteins by silencing leads to plants which are equipped with increased virus resistance and moreover, where the phenotype is essentially unchanged from wild type plants. With the present invention, it is also possible to show that the expression of dominant negative mutants of DnaJ-like proteins, which are no longer able to interact with viral factors or their cellular binding partners, allows the production of plants which are equipped with increased virus resistance. The method according to the invention, by means of which plants with increased virus resistance are produced by changing the expression or binding characteristics of DnaJ-like proteins, not only offers a useful alternative to the other methods of the prior art, but it also offers considerable advantages.

Because DnaJ-like proteins and the chaperone system of host cells in plants are involved in the infection process of plant viruses in a variety of ways, the method according to the invention offers the possibility of producing, e.g., transgenic plants which are resistant to a broad virus spectrum, i.e. resistant to both different virus groups and strains. The method according to the invention can be put into practice very efficiently and is generally applicable, as described below.

Depending upon whether several isoforms of DnaJ-like proteins or just specific DnaJ-like proteins are being silenced, and dependent upon the binding specificity of the viral components for the DnaJ-like proteins, plants can be produced which show resistance to a broad virus spectrum or to specific viruses. For example, the DnaJ-like proteins NtCPIP 1, NtCPIP2a and 2b, which have been more closely characterized within the framework of the invention, show specificity for components of the potyviruses PVY and TEV in the yeast two-hybrid system, whereas the DnaJ-like protein NtDnaJ-M541 preferably interacts with components from TSWV.

It is clear to the person skilled in the art that, in some cases, the duration of the resistance can also depend upon the type and number of the silenced DnaJ-like proteins (transient resistance) because, e.g. with the suppression of a single DnaJ-like protein, the viral components can possibly still interact with other isoforms of the DnaJ-like protein. In this case, the virus will be able to spread within the plant over a long period of time, despite transient resistance. However, there is also interest in plants which only exhibit transient virus resistance, as described below.

According to the invention, plant DnaJ-like proteins include proteins which are equipped with a J domain and are able to interact with viral components, i.e. viral (glyco-) proteins, viral nucleic acids and/or viral lipids. According to the invention, plant DnaJ-like proteins are also equipped with a J domain if an amino acid region of the plant protein shows significant homology to the consensus sequence of the J domain. Such homology is usually determined by means of alignment programs such as CLUSTAL. In general, the person skilled in the art can use appropriate algorithms to determine the sequence identity/similarity, e.g. with the program that is accessible under http://www.ncbi.nlm.nih.gov/BLAST (e.g., the link “standard nucleotide-nucleotide BLAST [blastn]”).

Another program which can be used for determining sequence homologies is ClustalW of the MegAlign program of DNASTAR Inc. (Madison, N.Y.).

DnaJ proteins have different structures and are grouped depending upon the combination of three additional and different domains (to the J domain), as originally identified in the E. coli orthologue: a gly/phe-rich domain, a cys-rich zinc finger domain and a less well conserved C-terminal domain which is possibly involved in substrate binding.

Dependent upon the combination of these domains, DnaJ-like proteins are arranged, e.g. in three groups I to III (Cheetham et al. (1998) Cell Stress Chaperones, 3, 28-36).

A comprehensive analysis of DnaJ-like proteins in Arabidopsis thaliana has led in addition to further classification with respect to the similarity of the J domain (Miemyk (2001) Cell Stress Chaperones, 6, 209-218).

The plant DnaJ-like proteins according to the invention, as identified within the framework of the present invention, form their own sub-class characterized by the single presence of a J domain. These plant DnaJ-like proteins are preferred for the production of plants and plant cells according to the invention. Other plant DnaJ-like proteins and their encoding sequences can also be used however. Similarly, non-plant DnaJ-like proteins and their encoding sequences can be used, e.g. when producing virus-resistant plants by means of the expression of dominant negative mutants.

Besides the DnaJ-like proteins and their homologues identified within the framework of the present invention, the proteins preferentially termed plant DnaJ-like proteins are proteins which belong to the phylogenetic strain 5 of the DnaJ-like proteins from Arabidopsis thaliana or their homologues (Miemyk et al., vide supra, page 213). Overall, 89 DnaJ-like proteins could be identified in Arabidopsis thaliana, of which the phylogenetic strain 5 includes seven members which all exhibit a predicted cytosolic localization. The members of this class as well as their homologues are also preferably termed plant DnaJ-like proteins within the framework of the present invention.

According to the invention, the preferred group of plant DnaJ-like proteins from tobacco (NtDnaJ-M541), tomato (Le19/8) and Arabidopsis (AtA39), recently identified as TSWV MP (NSm)-binding proteins, are lacking other DnaJ-typical domain structures, with the exception of the common J domain (Soellick et al. (2001) vide supra, von Bargen et al. (2001) vide supra). Moreover, a Lys-rich domain (4× K-X-X-X-K-E/K) could be identified which is also partially identifiable in the NtCPIP proteins (amino acids 172-177 and 269-274 for NtCPIP1 and amino acids 171-176 and 168-273 for NtCPIP2a and 2b) identified within the framework of the invention (K=lysine, E=arginine, X=any amino acid).

According to the invention, all plant proteins which are equipped with a J domain are thus termed DnaJ-like proteins. According to the invention, especially those proteins which only have one J domain and possibly the aforementioned lys-rich domain, are termed plant DnaJ-like proteins.

According to the invention, plant DnaJ-like proteins are particularly equipped with a J domain if they are equipped with amino acid sequences which are homologous to the three well conserved clusters of J domains which correspond to the secondary structural elements helix I, helix II and helix III. In particular, the HPD tripeptide of helix II as well as characteristic amino acids of helix III should be present (Kelley, 1998, TIBS, 23: 222-227).

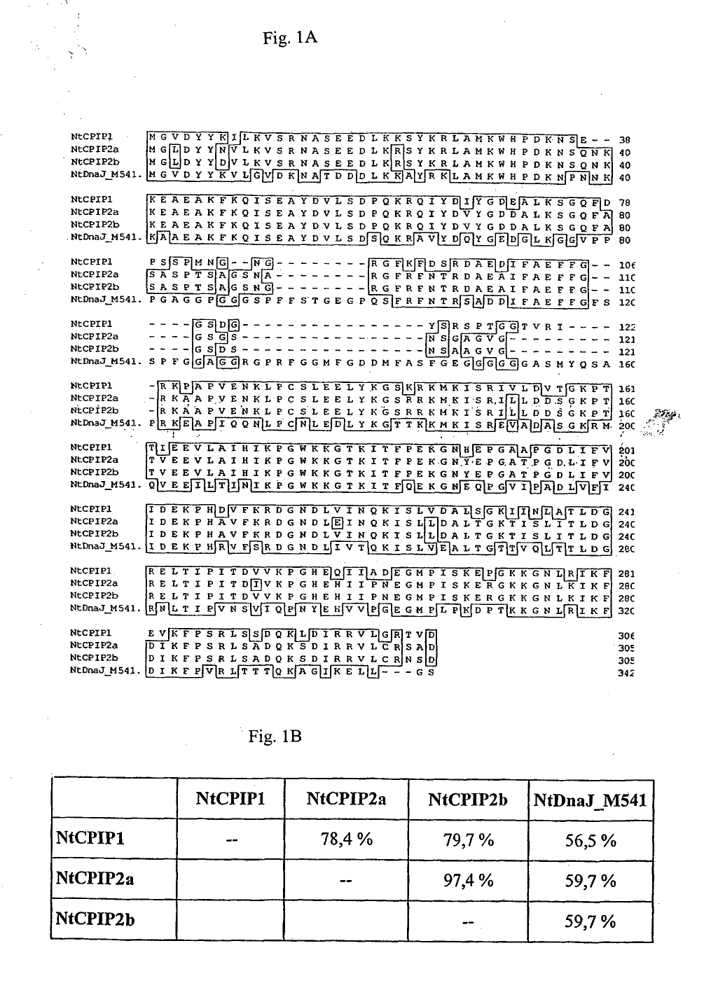

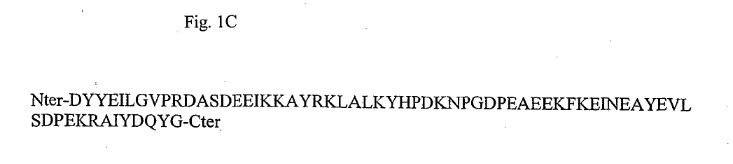

An alignment of several plant DnaJ-like proteins according to the invention, which have been proven to interact with viral components, is shown in FIG. 1. These are the proteins NtCPIP1, NtCPIP2a and NtCPIP2b from tobacco as well as NtDnaJ-M541 (Soellick et al., vide supra), which have been newly identified within the framework of the present invention. The characteristic sequence elements for the J domain are found at the N-terminus of the aforementioned proteins, i.e. within the first 70 amino acids.

A sequence characteristic for J domains is shown in FIG. 1c (SEQ ID NO:7). Regions of a protein show a significant homology to the sequence shown in SEQ ID NO:7 if the sequence of this region is at least 40%, preferably at least 50%, especially preferably at least 60%, also especially preferably at least 70% and/or 75%, particularly preferably at least 80%, 82%, 84%, 86% and/or 88% and most preferably at least 90%, 92%, 94%, 96%, 98% and/or 99% identical to the sequence with SEQ ID NO:7 (FIG. 1c). This is the DnaJ consensus sequence as found in the Conserved Domain Database of NCBI under Accession No: pfam00226.4.

In FIG. 1a the alignment of different J domains of different DnaJ-like proteins is shown as an example for the degrees of identity of different J domains. In FIG. 1b the identity (indicated as “percent identity”) and the distance (indicated as “divergence”) are shown. These are the proteins NtCPIP1, NtCPIP2a, NtCPIP2b and NtM541, Le 19-8 (NCBI Accession No: AJ295232), AtA39, (NCBI Accession No: AL021749, St_DnaJ (NCBI No: T07371), Nt_DnaJ (NCBI Accession No: CAC12824.1), At_DnaJ (NCBI Accession No: T49127), R1_DnaJ (NCBI Accession No: Y14649), Sc_DnaJ (NCBI Accession No: NP_{—}014335) and Ca_DnaJ (NCBI Accession No: P30725) characterized within the framework of the invention.

The greatest similarity to the plant DnaJ-like proteins NtCPIP1, 2a and 2e identified within the framework of the present invention is shown by various proteins from Arabidopsis thaliana. The gene bank accession numbers for the genes of these DnaJ-like proteins are AAD39315, AAS32885, AAF07844, AAD25656 and T48181. These proteins and their homologues are preferably termed plant DnaJ-like proteins within the framework of the present invention.

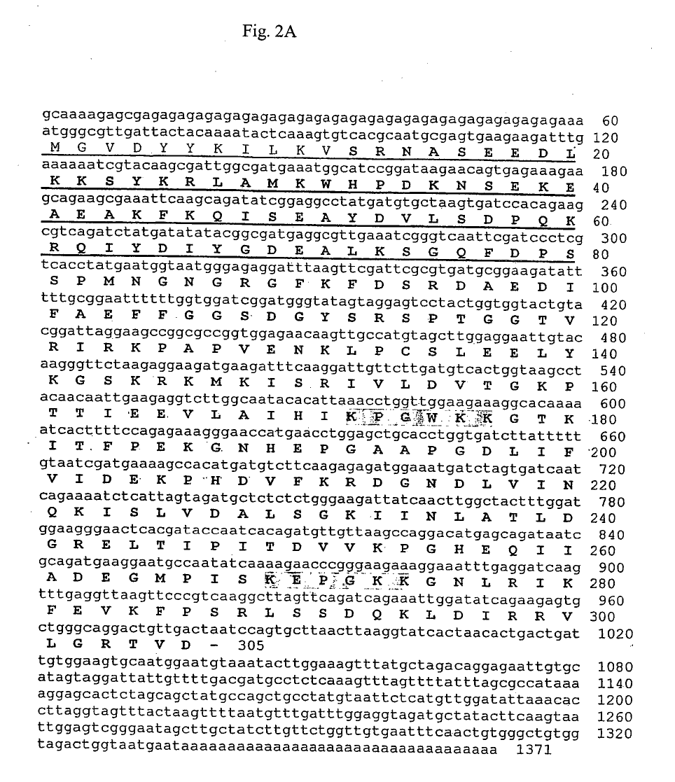

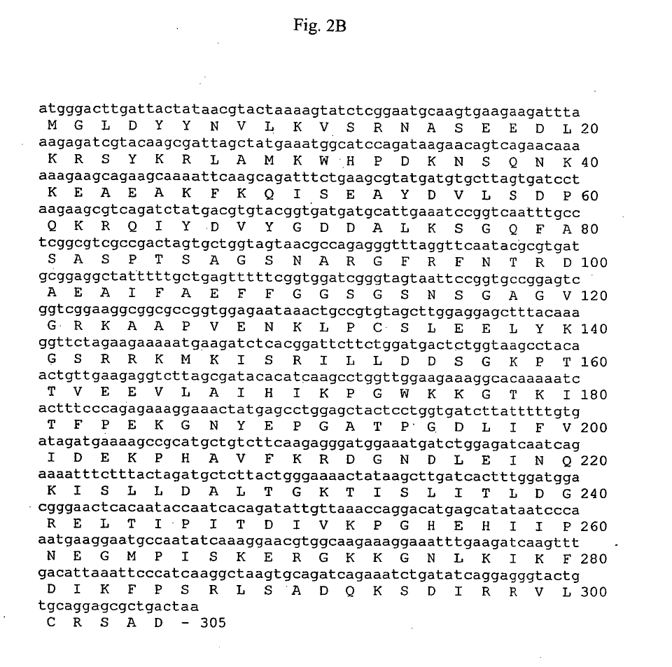

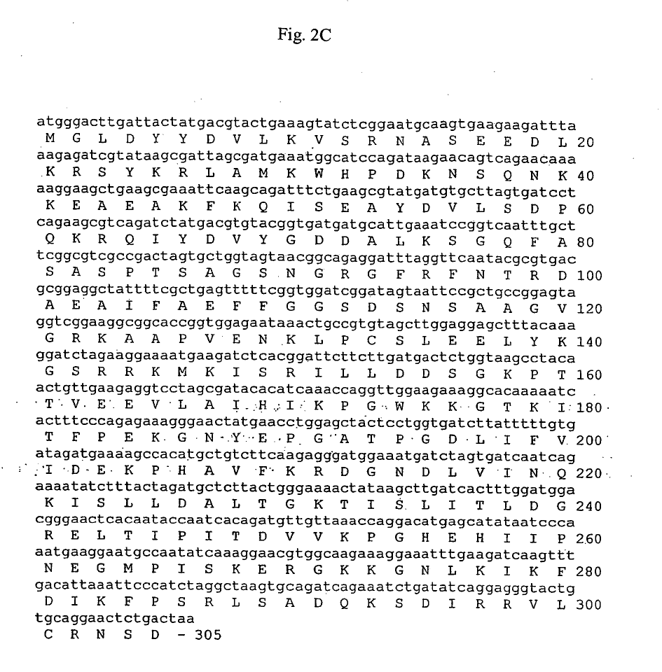

In particular, with plant DnaJ-like proteins whose suppression by silencing according to the invention can be used for the production of plants with increased virus resistance, one means proteins which have an amino acid sequence of sequence ID Nos. 4-6, or which are homologous to these (also see FIGS. 2a to 2c). Within the framework of the present invention, homology means that the proteins have an identity of at least 30%, preferably at least 40%, especially preferably at least 50%, also especially preferably at least 60%, particularly preferably at least 70% and/or 75% and most preferably at least 80%, 82%, 84%, 86% and/or 88% or at least 90%, 92%, 94%, 96%, 98% and/or 99% on the amino acid level. For the person skilled in the art it is obvious that proteins with a sequence identity of over 80% are homologous proteins.

The subject-matter of the instant invention includes, but is not limited to, plants and plants cells which have a transient or permanent virus resistance due to a modulation of the gene expression and/or the binding characteristics of plant DnaJ-like proteins. Modulating the expression of plant DnaJ-like proteins means that the expression of these proteins in comparison to corresponding wild type control plants is regulated upwards or downwards. With one of the preferred embodiments according to the invention, suppression of the expression of plant DnaJ-like proteins takes place in order to produce plants with increased virus resistance. In other cases the overexpression of plant DnaJ-like proteins can also be favored for the production of plants with increased virus resistance, in particular when dealing with dominant negative mutants of the DnaJ-like proteins.

Modulation of the binding characteristics of plant DnaJ-like proteins means that the plant DnaJ-like proteins have mutations which lead to the DnaJ-like proteins not being able, or only being able to a very limited extent, to interact either with viral factors, such as proteins, or with their physiological cellular binding partners. Such plants can e.g. be produced by overexpression of dominant negative mutants of DnaJ-like proteins if dealing with transgenic plants. Alternatively, dominant negative mutations can be introduced in the endogenous DnaJ-like proteins in order to obtain virus resistant plants.

The production of such plants and plant cells by the so-called “TILLING” method is described in greater detail below.

The method according to the invention can also be used to produce transgenic plants with increased virus resistance. Such plants can be resistant to different virus classes, groups and strains. Transgenic plants which are produced by a method according to the invention, are preferably resistant to poty, carla, hordei, potex, bunyai, clostero, cucumo, diantho, tobamo, gemini, lutero, rymo, tobra, furo, como, nepo, bromo and tospo viruses.

Particularly preferred are transgenic plants which are produced by the method according to the invention, and are resistant to potyvirus members, such as turnip mosaic virus (TuMV), tobacco vein mottling virus (TVMV), plum pox virus (PPV), bean common mosaic virus (BCMV), lettuce mosaic virus (LMV), zucchini yellow mosaic virus (ZYMV), potato virus A (PVA), potato virus Y (PVY) and tobacco etch virus (TEV).

Equally preferred are transgenic plants according to the invention which are resistant to members of the tospoviruses, such as bromo-, chomo- and caulimoviruses. Particularly preferred are transgenic plants according to the invention that are resistant to the cowpea mosaic virus (CPMV), the tomato spotted wilt virus (TSWV) as well as the cauliflower mosaic virus (CaMV).

The transgenic plants from the present invention are particularly resistant to the potyvirus viral strains PVY^{N}, PVY⁰, PVY^{T}, and PVY^{C}, as well as the potato tuber necrotic isolates (PVY^{NTN}).

According to the invention, transgenic plants in which a specific plant DnaJ-like protein was silenced are not necessarily resistant to all mentioned virus classes, groups and strains. These transgenic plants will rather be particularly resistant to viruses, the components of which interact with the plant DnaJ-like protein during the infection cycle. Because the same virus components of different virus groups often interact with the same DnaJ-like protein, transgenic plants which are resistant to a relatively broad virus spectrum can be produced with methods according to the invention.

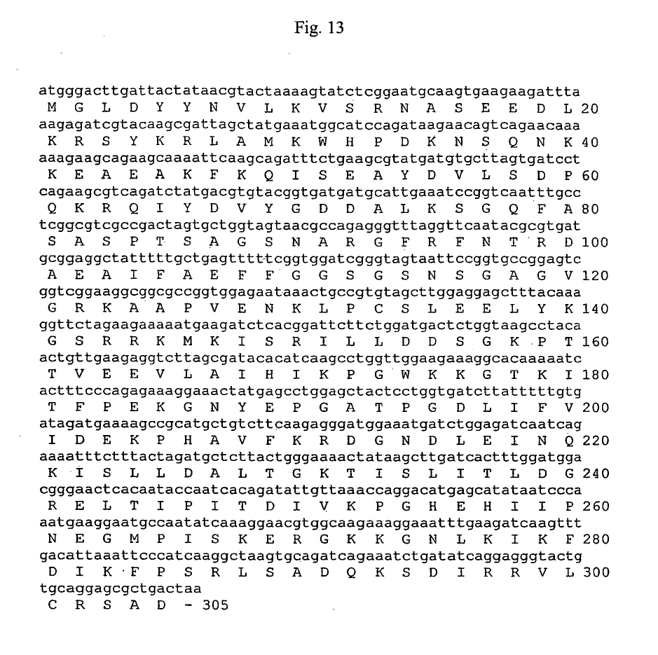

In this way, transgenic plants which are resistant to the PVY virus and the TEV virus can be produced by methods according to the invention, e.g. by the silencing of the DnaJ-like protein which is encoded by SEQ ID NO. 1 (NtCPIP1, see FIG. 2a). This is justified by the fact that the capsid proteins of these viruses interact with the DnaJ-like protein of SEQ ID NO. 4 (NtCPIP1, see FIG. 2a). The same correspondingly applies to proteins which are encoded by SEQ ID NO:2 (NtCPIP2a, see FIG. 2b), SEQ ID NO:3 (NtCPIP2b, see FIG. 2c) and SEQ ID NO: 10 (FIG. 13), and which have the amino acid sequences of SEQ ID NO:5 (NtCPIP2a, see FIG. 2b) and SEQ ID NO:6 (NtCPIP2b, see FIG. 2c).

The subject-matter of the invention therefore also relates to plant DnaJ-like proteins with the amino acid sequences of NtCPIP1, 2a and 2b (SEQ ID Nos. 4, 5 and 6) and nucleic acid sequences which encode these proteins and their homologues, as well as their use for the production of transgenic plants.

Because virus components important for the infection cycle are possibly also able to interact with different cellular DnaJ-like proteins (e.g. with different isoforms), it can be necessary in such cases to silence several DnaJ-like proteins (e.g. the different isoforms) in order to achieve lasting virus resistance of the plants. In said cases, the silencing of only one specific DnaJ-like protein can lead to transient resistance. It is described below how several DnaJ-like proteins can be silenced.

Even transient silencing or transient resistance can be advantageous because it is a valuable addition to other methods which allow the production of plants with increased virus resistance, but which affect the phenotype. Because plants which are produced by methods according to the invention and which show transient resistance during development of the virus infection do not exhibit any significant change of phenotype, methods according to the invention which give rise to transient resistance can help, along with other methods, to produce plants which are characterized by more enduring and more stable virus resistance, without having any negative effect upon the phenotype restriction caused by the methods.

The methods according to the invention can also be put into practice to produce transgenic plant cells which have increased virus resistances.

In addition to transgenic plant cells, the subject-matter of the invention includes transgenic plants obtained by regeneration of plant cells according to the invention. Transgenic plants and the transgenic plant cells can signify any monocotyledonous or dicotyledonous plant or plant cell, preferably agricultural plants or cells from agricultural plants. Particularly preferred are plants with storage organs, such as potato. Equally preferred are sugar-beet, tomato, banana, carrot, sugar cane, corn, strawberry, pineapple and papaya.

The invention also relates to the crop products and propagation material of transgenic plants, the cells of which have increased virus resistance. Crop products and propagation material involve in particular, fruit, seeds, tubers, rootstocks, germs, cuttings etc. They can also involve parts of these plants, such as plant cells, protoplasts and calli.

The subject-matter of the invention thus also includes transgenic plants which are produced by the methods according to the invention, and which have the nucleic acid molecules described below integrated into the plant genome or which contain these as autonomously replicating molecules. Autonomously replicating virus vectors such as PVX (Ruiz et al., 1998, Plant Cell, 10: 937-946) or TRV (Ratcliff et al., 2001, Plant J., 25, 237-245) which carry specific sequences for plant DnaJ-like proteins can e.g., be used in order to silence respective DnaJ-like proteins by means of the principle of “virus-induced gene silencing” (VIGS).

In principle, any plant can be utilized. It is preferably a monocotyledonous or dicotyledonous agricultural, food or feed plant. Examples of monocotyledonous plants are plants which belong to the genera Avena (oat), Triticum (wheat), Secale (rye), Hordeum (barley), Oryza (rice), Panicum, Pennisetum, Setaria, Sorghum (millet), Zea (maize) and the like. Dicotyledonous agricultural plants include, among others, cotton, legumes such as pulses and in particular alfalfa, soya bean, rape, tomato, sugar-beet, potato, ornamental plants and trees. Further agricultural plants can include fruit (in particular apples, pears, cherries, grapes, citrus fruits, pineapples and bananas), oil palms, tea, cocoa and coffee trees, tobacco, sisal, as well as medical plants such as rauwolfia and digitales. Particularly favored are the cereals wheat, rye, oat, barley, rice, maize and millet, sugar beet, rape, soya, tomato, potato and tobacco. Other agricultural plants can be taken from U.S. Pat. No. 6,137,030.

The plant cells according to the invention include differentiated and undifferentiated plant cells including protoplasts which were produced by the methods according to the invention and which have integrated the nucleic acid molecules described in the following into the plant genome, or have received them as autonomously replicating molecules.

According to the invention, transgenic plants which are equipped with increased virus resistance can be produced by the expression of plant DnaJ-like proteins being essentially suppressed. Within the framework of the present invention “essentially suppressed” and “substantially prevented” mean that the level of expression of DnaJ-like proteins in transgenic plants is reduced to a level at which the plants are resistant to viral infections and/or show signs of recovery after the initial symptoms of a viral infection. Within the framework of the present invention, “virus resistant transgenic plants” also means plants which only show slight symptoms of a viral infection, but which are, however, so slight that the applicability and usefulness of the affected plants is not questioned. Within the framework of the present invention, “transiently virus resistant transgenic plants” means plants which, for a certain period—typically at the beginning of a viral infection—show clear symptoms of virus resistance. Plants according to the invention show an essentially unaltered phenotype, so that, e.g., the use of such plants as agricultural, food or feed plants etc. is not questioned.

With the methods for the production of transgenic plants with increased virus resistance according to the invention, the suppression of the expression of plant DnaJ-like proteins can be achieved by means of different strategies. The expression of plant DnaJ-like proteins can be essentially suppressed in transgenic plants e.g. by silencing. With silencing, a nucleic acid which encodes plant DnaJ-like proteins or fragments thereof and/or which is complementary to it, is transferred to the plant. In order to ensure that the plants are transgenic for the transferred nucleic acids, the nucleic acid to be transferred is generally transferred to the plant by means of a vector, such as a plasmid, which is able to stably replicate within the plant cell or to integrate the transferred nucleic acid into the plant genome.

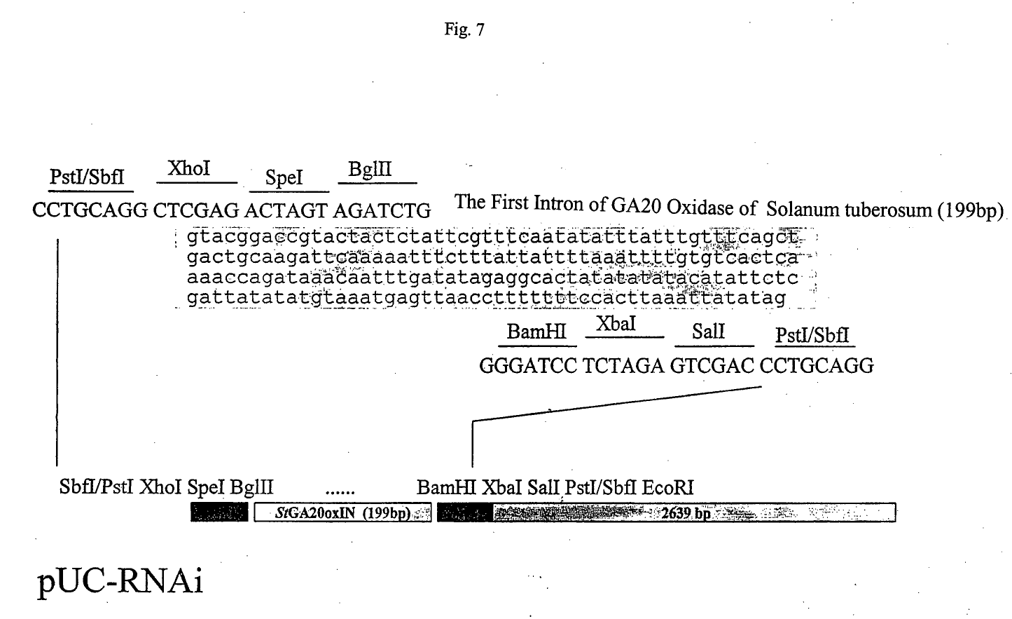

The RNAi method can be preferably used for the silencing of plant DnaJ-like proteins. In so doing, a vector which includes the following elements in 5′-3′ direction is transferred to the plant cell: a promoter which is functional in plants; operatively linked to this, a DNA sequence which includes the antisense sequence of the sequence encoding the plant DnaJ-like protein or fragments thereof and which has 3′exon sequences at its 3′end recognizable by the spliceosome; an intron; a DNA sequence which includes the sense sequence of the DNA sequence encoding the plant DnaJ-like protein or fragments thereof and which has 5′exon sequences at it 5′end recognizable by the spliceosome; and a termination sequence. A vector of this type is illustrated in FIG. 7. The position of the antisense and sense sequences can, of course, be exchanged. It is clear to the person skilled in the art that the respective 5′ and 3′ splice sites must be correspondingly adapted.

If vectors of this type are stably transferred to plant cells, the transcription of these vectors gives rise, first of all, to a pre-mRNA which consists of a first exon which includes the antisense sequence of the sequence encoding plant DnaJ-like proteins or fragments thereof, an intron and a second exon which includes the sense sequence of the DNA sequence encoding plant DnaJ-like proteins or fragments thereof. Because the intron is removed by the splicing process, a continuous RNA molecule emerges with regions which are complementary to each other. This type of RNA molecule will develop a double-stranded structure (Smith et al., 2000, Nature, 407:319-320).

These double-stranded RNA molecules are able to specifically silence the mRNA of plant DnaJ-like proteins by induction of the PTGS system so that consequently, plant DnaJ-like proteins are no longer expressed. Exactly which plant DnaJ-like proteins are no longer expressed can be determined by the appropriate choice of antisense and sense sequences. Dependent upon whether these antisense and sense sequences only include the sequence encoding the J domain or fragments thereof, or include sequences which are characteristic for the DnaJ protein in question, it is therefore possible to silence either a multitude of DnaJ-like proteins or just very specific DnaJ-like proteins. The sequences characteristic for a protein can be determined by sequence homology programs, as described above for the amino acid regions characteristic for the J domain. Such determinations are within the standard knowledge of those skilled in the art. Those skilled in the art additionally know that a multitude of plant DnaJ-like proteins can be silenced by the use of their respective characteristic sequences.

It is also known in the art that, besides the vectors mentioned, other vectors can also be used for the RNAi method or PTGS. These vectors can be produced, e.g. so that the respective sense and antisense sequences can be transcribed starting from a U6 promoter, hybridize in the cell and induce the PTGS system (Tuschl, 2002, Nat. Biotechnol. 20, 446-448; Miyagishi et al., 2002, Nat. Biotechnol., 20, 497-500; Lee et al., 2002, Nat. Biotechnol., 20, 500-505). With other vectors, the sense and antisense sequences are linked by a loop sequence and are transcribed by a human RNAse P RNA H1 promoter. By folding back the loop, the sense and antisense sequences can hybridize, form double-stranded RNA, and induce the PTGS system (Tuschl, 2002, vide supra; Paul et al., 2002, Nat. Biotechnol., 20, 505-508; Paddison et al., 2002, Genes Dev., 16, in press, Brummelkamp et al., 2002, Science, 296, 550-553).

With another embodiment of the invention, the vectors used to transfer the nucleic acids include in 5′-3′-orientation a promoter, a DNA sequence operatively linked to this which includes the antisense sequence of the sequence encoding plant DnaJ-like proteins or fragments thereof, and a termination sequence. With the transcription of these vectors in plant cells, an RNA molecule emerges, the sequence of which is complementary to the sequence encoding plant DnaJ-like proteins or fragments thereof. By hybridizing the antisense sequence with endogenous mRNA sequences of plant DnaJ-like proteins in vivo, the expression of plant DnaJ-like proteins can thus be suppressed in plant cells.

In an additional embodiment, the vectors used to transfer the nucleic acids include, in 5′-3′ orientation, a promoter, an operatively linked DNA sequence which includes the sequence encoding plant DnaJ-like proteins or fragments thereof and which contains self-complementary sections, and a termination sequence. With the transcription of these vectors in the plant cell, RNA molecules emerge which are equipped with sequence regions that can hybridize with each other. By these means, double-stranded RNA molecules can occur in the cell that induce the PTGS system, and leading to the mRNA of the respective DnaJ-like proteins being specifically degraded. The process also known as co-suppression for the silencing of plant proteins presupposes that the mRNA of the plant DnaJ-like protein which is to be suppressed is equipped with sections that are complementary to one another. These sections can be identified by those skilled in the art by simply making a visual inspection of the DNA sequence encoding the protein in question or by appropriate sequence programs, such as DNAStar of DNASTAR Inc., Madison, USA.

With another embodiment of the methods according to the invention, the vectors used for the transfer of the nucleic acids to the plant cells include in 5′-3′-orientation promoters which are functional in plants, a DNA sequence operatively linked to this which encodes a ribozyme which specifically recognizes the mRNA of plant DnaJ-like proteins, and a termination sequence. Those skilled in the art is aware how ribozymes which have an endonuclease activity, aimed at a specific mRNA, can be produced. This is described in detail e.g. in Steinecke et al., 1992, EMBO J., 11: 1525. Within the framework of the present invention, the term “ribozymes” also means those RNA sequences which, besides the actual ribozyme, also include leader sequences that are complementary to the mRNA of the DnaJ-like proteins or fragments thereof, and therefore lead the mRNA-specific ribozyme more specifically to the mRNA substrate of the ribozyme.

Another alternative for the production of transgenic plants with increased virus resistance is offered by the transfer of nucleic acids by means of vectors which include in 5′-3′-orientation, promoters which are functional in plants, a DNA sequence operatively linked to this which includes the antisense sequences of the sequences encoding the plant DnaJ-like proteins or fragments thereof, the sequence encoding RNAse P, and a termination sequence. With the transcription of such vectors, RNA molecules emerge in the cell that are equipped with a leader sequence (the antisense sequence) which leads the RNAse P to the mRNA of the DnaJ-like protein, whereby the cleavage of the mRNA is brought about by RNAse P (U.S. Pat. No. 5,168,053). The leader sequence preferably includes 10 to 15 nucleotides, which are complementary to the DNA sequence of the DnaJ-like protein, and a 3′-NCCA nucleotide sequence, where N is preferably a purine. The transcripts of the external leader sequence bind to the target mRNA by forming base pairs, which allows the cleavage of the mRNA by means of the RNAase P at the nucleotide 5′ from the paired region. An mRNA cleaved in this way cannot be translated into a functional protein.

Moreover, for the production of transgenic plants with increased virus resistance according to the invention, vectors can also be used which have a DNA sequence with the following constituents in 5′-3′-orientation: a DNA sequence which corresponds to the 5′ region of the DNA sequence encoding DnaJ-like proteins, a DNA sequence for resistance genes as well as a DNA sequence which corresponds to the 3′-region of the sequence encoding plant DnaJ-like proteins. These vectors can be used in order to bring about a specific gene knock-out of the plant DnaJ-like proteins of note by means of homologous recombination. With plant cells in which the homologous recombination has taken place, the sequence for the resistance gene is inserted into the DNA which encodes the plant DnaJ-like protein, so that no more functional mRNA of the plant protein similar to DNA can be produced in the cell. By selection against the resistance gene, the plant cells, in which the recombination has taken place, can be identified. The person skilled in the art is aware of how these vectors for gene knock-out by means of homologous recombination are constructed, which elements they must contain (promoters, enhancers, flanking sequences) and how the knock-out plant cells are identified. Typically, antibiotic resistance genes are used as resistance genes. Other resistance genes which allow a selection of the cells in which the recombination has taken place can also be used.

If nucleic acid sequences that encode plant DnaJ-like proteins or fragments thereof are mentioned within the framework of the present invention, the complete coding DNA sequence of the respective plant DnaJ-like protein in question is meant, as well as the complete mRNA sequence and the respective fragments. Because some of the afore-mentioned methods for the production of transgenic plants, in which the expression of plant DnaJ-like proteins is significantly reduced, are based upon the fact that a specific hybridization between the endogenous mRNA of the plant DnaJ-like proteins and the sequences which emerge with the transcription of the aforementioned vectors (such as the antisense strategy), the person skilled in the art knows that the transferred nucleic acids need not always contain the whole sequence encoding DnaJ-like proteins, whether dealing with the sense or antisense sequence. In fact, for a specific hybridization, relatively small regions of the sequences coding for DnaJ-like proteins can be sufficient for efficient silencing.

With vectors, the transcription of which leads to double-stranded RNA molecules, it is sufficient if the sequences corresponding to the sequence regions of the mRNA of plant DnaJ-like proteins include around 25 nucleotides. The transferred sequences generally include between 25 and 1000 nucleotides, preferably between 25 and 750 nucleotides, especially preferably around 400 to 800 and 500 to 750 nucleotides. Sequences can also be used, however, which include between 25 and 500 nucleotides, between 25 and 300 nucleotides, between 25 and 150 nucleotides and between 25 and 100 nucleotides.

The person skilled in the art knows that, with the RNAi or PTGS, the sense and antisense RNAs used to form double-stranded RNA molecules can also include around 21 to 23 nucleotides with a characteristic 3′ overhang (Tuschl, 2002, Nat. Biotechnol. 20, 446-448).

When nucleic acids are transferred to plant cells, the transcription of which leads to sequences in the cell that are complementary to the mRNA of the plant DnaJ-like proteins (e.g. with the antisense strategy), the sequences need not be one hundred percent complementary to the mRNA. In fact, it is sufficient if these sequences are at least 50% complementary, preferably at least 60%, especially preferably at least 70%, also especially preferably at least 80%, particularly preferably at least 90% and most preferably at least 95%. Deviations can arise as a result of deletion, addition, substitution and/or insertion. It is clear to the person skilled in the art that, with reduced complementarity, the probability increases that several DnaJ-like proteins are silenced.

It is generally the case that only those complementary sequences can be used according to the invention that are able to hybridize specifically with the mRNA regions of the plant DnaJ-like proteins. Sequences that hybridize in vivo with RNA regions of proteins other than plant DnaJ-like proteins and cause their silencing are not suitable for the methods according to the invention. Depending upon the sequence chosen, i.e. whether the sequence corresponds to the J domain or to the sequence regions characteristic for the DnaJ-like protein in question, and depending upon the degree of complementarity, a multitude or just a few plant DnaJ-like proteins are silenced. Possibly, only the expression of a very specific DnaJ-like protein is suppressed. The length of complementary sequences is preferably between 20 and 1000 nucleotides, also preferably between 20 and 750 nucleotides, especially preferably between 20 and 500 nucleotides, also especially preferably between 20 and 300 nucleotides, particularly preferably between 20 and 150 nucleotides, also particularly preferably between 20 and 75 nucleotides, and most preferably around 20 and 50 nucleotides. Possibly, the sequences can only include around 20 or 25 nucleotides.

Some of the aforementioned methods can also be carried out with sequences that are not a constituent of the coding part of the mRNA of plant DnaJ-like proteins or are complementary to the same. It can e.g. be sufficient to have sequences from the 5′- or 3′-untranslated region, provided that these regulatory sequences are characteristic for the mRNA of the DnaJ-like protein or the DnaJ-like proteins.

Such sequences can be used in particular if the silencing is induced by double-stranded RNA constructs or the translation of an mRNA is inhibited by antisense constructs. According to the invention, the term mRNA therefore covers not only the coding components of the mRNA of plant DnaJ-like proteins, but also all regulatory sequences which occur in the pre-mRNA or mature mRNA and which are characteristic for the mRNA of DnaJ-like proteins. This also applies to the DNA sequences as well, which can involve e.g. 5′- and 3′-untranslated regions, promoter sequences, upstream activating sequences, introns, etc.

If vectors are used, the transcription of which leads to RNA molecules that consist of a leader sequence and RNAse P, the leader sequence must be sufficiently complementary so as to specifically recognize plant DnaJ-like proteins. The region of the mRNA of the plant DnaJ-like proteins recognized by the leader sequence can be chosen according to the requirements in question. Preferably, such leader sequences include around 20 nucleotides, but they should not, however, be significantly shorter than 15 nucleotides. With 100% complementarity of the leader sequence, 12 nucleotides should also be sufficient. Of course, the leader sequences can include up to 100 nucleotides or more because in this way their specificity for the mRNA in question is merely increased.

When the term sense sequences is mentioned within the framework of the present invention, it refers to those sequences which correspond to the coding strand of the genes for plant DnaJ-like proteins or fragments thereof. These sequences must not, however, be 100% identical to the sequences encoding the plant DnaJ-like proteins which are of interest. It is sufficient if the sequences are sufficiently similar to the sequences encoding DnaJ-like proteins, so that their expression in plant cells leads to an efficient and specific silencing of plant DnaJ-like proteins in the cell e.g. by RNA interference or co-suppression. It should be sufficient if these sequences are at least 50% identical, preferably at least 60%, especially preferably at least 70%, also especially preferably at least 80%, particularly preferably at least 90% and most preferably at least 95%. Such degrees of identity according to the invention are established so that the sequences are homologous to one another or show homology. Deviations to the sequence encoding DnaJ-like proteins or fragments thereof can arise as a result of deletion, addition, substitution and/or insertion. It is clear to the person skilled in the art that as identity decreases the probability increases that several DnaJ-like proteins will be silenced. Sequences, the degree of identity or homology of which is so low that proteins other than plant DnaJ-like proteins are silenced, are not sufficiently specific and not suitable for the methods according to the invention.

The person skilled in the art is familiar with the terms complementarity, homology and identity.

According to the invention, sequence homology and homology generally mean that the nucleic acid or the amino acid sequence of a DNA molecule or a protein is at least 40% identical to the nucleic acid or amino acid sequences of a known DNA or RNA molecule or protein, preferably at least 50%, more preferably at least 60%, also more preferably at least 70% and/or 75%, especially preferably at least 80%, 82%, 84%, 86% and/or 88%, particularly preferably at least 90%, 92% and/or 94% and most preferably at least 95%, 96%, 97%, 98% and/or 99%.

The term complementarity describes the ability of a nucleic acid molecule to hybridize with another nucleic acid molecule using hydrogen bonds between complementary bases. The person skilled in the art knows that two nucleic acid molecules need not have 100 percent complementarity in order to hybridize with one another. A nucleic acid sequence which will hybridize with another nucleic acid sequence is preferably at least 40% complementary, at least 50%, at least 60%, preferably at least 70%, especially preferably at least 80%, also especially preferably at least 90%, particularly preferably at least 95%, and most preferably at least 98 or 100%.

Nucleic acid molecules are identical if they have the same nucleotides in the same 5′-3′-orientation.

Similarly, antisense sequences according to the invention include sequences that correspond to the non-coding DNA strand of the gene of the plant DnaJ-like proteins which are of interest. Of course these sequences need not be one hundred percent identical with the sequence of the non-coding DNA strand of the genes of the DnaJ-like proteins which are of interest, but rather can show the aforementioned degrees of homology. This circumstance is also expressed by the fact that antisense sequences which, by definition, are complementary to the mRNA of a gene, must not be 100% complementary to the mRNA. They can, e.g. also be at least 50% complementary, preferably at least 60% complementary, especially preferably at least 70% complementary, also especially preferably at least 80% complementary, particularly preferably at least 90% complementary and most preferably at least 95%, 98% and/or 100% complementary. As stated above, it is sufficient if the antisense sequences are able to hybridize specifically with the mRNA of the plant DnaJ-like proteins which are of interest. The hybridization can either take place in vivo under cellular conditions or in vitro.

The hybridization of an antisense sequence with an endogenous mRNA sequence typically takes place in vivo under cellular conditions, or in vitro.

According to the invention, hybridization in vitro is always carried out under conditions sufficiently stringent to guarantee a specific hybridization. Such stringent hybridization conditions are known to the person skilled in the art and are described in the literature (see e.g., Sambrook et al., 2001, Molecular cloning: A laboratory manual, 3^{rd} edition, Cold Spring Harbor Laboratory Press).

In general, to “specifically hybridize” means that a molecule preferentially binds under stringent conditions to a specific nucleotide sequence if this sequence is in a complex mixture of (e.g. total) DNA or RNA. The term “stringent conditions” generally refers to conditions under which a nucleic acid sequence will preferentially hybridize to its target sequence and to a considerably lesser extent or not at all to other sequences. Stringent conditions are partly sequence-dependent and will be different under different conditions. Longer sequences hybridize specifically at higher temperatures.

In general, stringent conditions are chosen so that the temperature is approx. 5° C. below the thermal melting point (Tₘ) for the specific sequence with a defined ion strength and a defined pH. The Tₘ is the temperature (under defined ion strength, pH and nucleic acid concentration) at which 50% of the molecules complementary to the target sequence hybridize to the target sequence in a state of equilibrium. Typically, stringent conditions are those under which the salt concentration is at least approx. 0.01 to 1.0 M sodium ion concentration (or another salt) with a pH of between 7.0 and 8.3, and the temperature is at least approx. 30° C. for short molecules (that is e.g. 10-50 nucleotides). In addition, stringent conditions, as mentioned above, can be achieved by adding destabilizing agents, such as formamide, for example.

Typical hybridization and wash buffers have, e.g. the following composition:

Prehybridization Solution:

0.5% SDS
5×SSC
50 mM NaPO₄, pH 6.8
0.1% Na pyrophosphate
5× Denhardt's solution
100 μg/ml salmon sperm
   Hybridisation Solution:
Prehybridization solution
1×10⁶ cpm/ml probe (5-10 min 95° C.)
   20×SSC:
3 M NaCl
0,3 M sodium citrate
ad pH 7 with HCl
   50× Denhardt's Reagent:
5 g Ficoll
5 g polyvinyl pyrrolidone
5 g bovine serum albumine
ad 500 ml A. dest.

A typical method for hybridization is as follows:
Optional: wash blot 30 min in 1×SSC/0.1% SDS at 65° C.
Prehybridization: at least 2 hrs at 50-55° C.

Hybridization: over night at 55-60° C.

| | | | |
|---|---|---|---|
| Wash: | 05 min | 2x SSC/0.1% SDS | hybridization temp. |
| | 30 min | 2x SSC/0.1% SDS | hybridization temp. |
| | 30 min | 1x SSC/0.1% SDS | hybridization temp. |
| | 45 min | 0.2x SSC/0.1% SDS | 65° C. |
| | 5 min | 0.1x SSC | room temperature |

The person skilled in the art knows that the specified solutions and the protocol shown can or must be modified dependent upon the application.

The person skilled in the art is also acquainted with the terms “sense” and “antisense”. The person skilled in the art of silencing genes in plants also knows from the state of the art how long the nucleic acid molecules to be used for the silencing must be and which homology and complementarity they must have to the sequences which are currently of interest. According to the invention, anti-sequences, which e.g. cannot hybridize specifically with coding sense sequences of plant DnaJ-like proteins in vivo and/or in vitro, i.e. which also hybridize with the coding sense sequences of other protein classes, cannot be used.

The vectors used for the silencing of plant DnaJ-like proteins include regulatory elements in addition to the nucleic acid sequence to be transferred. Exactly which regulatory elements these vectors should contain depends upon the respective method with which the vectors are used. Those skilled in the art practicing the aforementioned methods for the production of transgenic plants in which the expression of a protein is suppressed, are aware of which regulatory elements and other elements these vectors must contain.

Typically the regulatory elements which the vectors contain are those which guarantee transcription and, if so desired, translation within the plant cell.

The nucleic acid sequences to be transferred can therefore, for example, be under the control of promoters which are functional in plants. These promoters can be constitutive, inducible, tissue- or development-specific promoters. Moreover, they can also be virus-specific promoters. In this way e.g. transgenic plants can be produced which, under normal circumstances, express the plant DnaJ-like proteins, but if attacked by a virus, silence the genes for DnaJ-like proteins by means of the virus-specific promoter in the cells first affected.

Typically, the constitutive 35S promoter will be used as a promoter for vectors. Moreover, other promoters can be used which are obtained from different sources, such as plants or plant viruses, and which are suitable for the expression of genes in plants. The choice of promoter and of other regulatory sequences determines the local and temporal expression pattern and also the silencing of the plant DnaJ-like proteins in transgenic plants.

Besides additional constitutive promoters, such as the actin promoter (McElroy et al., 1990, Plant Cell, 2:163) and the ubiquitin promoter (Binet et al., 1991, Plant Science, 79:87), the tissue-specific promoters of the phosphoenol pyruvate carboxylase from corn (Hudspeth et al., 1989, Plant Mol. Biol., 12:579) or of the fructose 1,6-bisphosphatase from potato (WO98/18940), which determine the leaf-specific expression, can also be considered. Wound-, light- or pathogen-induced and other development-dependent promoters or control sequences can also be used (Xu et al., 1993, Plant Mol. Biol. 22:573; Logemann et al., 1989, Plant Cell, 1:151; Stockhaus et al., 1989, Plant Cell, 1:805; Puente et al., 1996, EMBO J., 15:3732; Gough et al., 1995, Mol. Gen. Genet., 247:323). A summary of useable control sequences can be found, e.g. in Zuo et al., 2000, Curr. Opin. Biotech., 11:146.

Appropriate promoters also include promoters which guarantee an expression solely in photosynthetically active tissues, e.g. the ST-LS1 promoter (Stockhaus et al. (1987) Proc. Natl. Acad. Sci. USA 84: 7943-7947; Stockhaus et al. (1989) EMBO J. 8:2445-2451). Promoters can also be used which are active during plant transformation, plant regeneration or specific stages of these processes, such as cell division-specific promoters such as the Histone H3 promoter (Kapros et al. (1993) InVitro Cell Cev. Biol. Plant 29:27-32) or the chemically induceable Tet-repressor system (Gatz et al. (1991) Mol. Gen. Genet. 227:229-237). Other suitable promoters are described in the literature, e.g. Ward (1993, Plant Mol. Biol. 22:361-366). Similarly, induceable and cell- or tissue-specific promoters, such as meristem-specific promoters, are also described in the literature and are suitable within the framework of the invention.

Other induceable promoters include virus-induceable promoters such as the ACMV virion sense promoter (Hong et al., 1996, Virology, 220:119-227) which is induced by the gene product AC2. Moreover, all promoters of such proteins that are induced in virus-infested tissues, such as phenylalanine ammonium lyase, chalcone synthase, hydroxyproline-rich glycoprotein, extensin, pathogenesis-related proteins (e.g. PR-1a) and wound-induceable protease inhibitors (U.S. Pat. No. 6,013,864), are suitable.

Moreover, the average person skilled in the art is able to isolate additional suitable promoters by means of routine methods. The person skilled in the art, with the help of established molecular biology methods, e.g. hybridization experiments or DNA-protein-binding studies, can thus identify storage organ-specific regulatory nucleic acid elements. In so doing, e.g., in a first step the whole poly(A)⁺-RNA is isolated from the storage organ tissue of the required organism from which the regulatory sequences are to be isolated, and a cDNA library is generated. In a second step, and with the help of cDNA clones based on poly(A)⁺-RNA molecules from a non-storage organ tissue, those clones, the corresponding poly(A)⁺-RNA molecules of which only accumulate in the tissue of the storage organ, are identified from the first library by means of hybridization. Finally, with the help of cDNAs identified in this way, promoters are isolated that are equipped with storage organ-specific regulatory elements. Other methods based on PCR are available to the person skilled in the art for the isolation of appropriate storage organ-specific promoters.

Another embodiment uses the promoter of the class I patatin gene B33 from potato. Other favored promoters are those that are particularly active in fruits. These include, for example, the promoter of a polygalacturonase gene, e.g. from tomato, which mediates expression during the maturation of tomato fruits ((Nicholass et al.) (1995) Plant Mol. Biol. 28:423-435; describing the analysis of promoter/GUS fusion constructs), the promoter of an ACC oxidase, e.g. from apple, which mediates maturity and fruit specificity in transgenic tomatoes (Atkinson et al. (1998) Plant Mol. Biol. 38:449-460; describing promoter/GUS expression analyses), or the 2A11 promoter from tomato (van Haaren et al. (1991) Plant Mol. Biol. 17:615-630, also describing promoter/GUS fusions).

Also in the case of fruit-specific promoters, the person skilled in the art can use other suitable promoters described in the literature, or as described above for storage organ-specific promoters, isolate them using routine methods.

The person skilled in the art knows that the use of induceable promoters allows the production of plants and plant cells which only transiently express, and so only transiently silence the sequences according to the invention. Such a transient expression allows the production of plants which only show transient virus resistance. Such a transient resistance can e.g. be desirable if there is the risk of virus contamination and the plants therefore need to be resistant to the virus only for a particular length of time. The person skilled in the art is aware of other situations in which transient resistance is desirable. The person skilled in the art is also aware that, by the use of vectors which do not stably replicate in plant cells and which carry the respective sequences for the silencing of plant DnaJ-like proteins, he can achieve transient expression and therefore also transient silencing and transient resistance.

The vectors according to the invention can also include other e.g. enhancer elements as regulatory elements. In addition they can contain resistance genes, replication signals and other DNA regions which allow the propagation of the vectors in bacteria such as E. coli. The regulatory elements also include sequences which bring about stabilization of the vectors in the host cells. In particular, these regulatory elements include sequences that allow stable integration of the vector into the plant's host genome or an autonomous replication of the vector in the plant cells. The person skilled in the art is acquainted with this type of regulatory elements.

Termination sequences refer to sequences that ensure that the transcription or the translation is properly terminated. If the transferred nucleic acids are to be translated, they are typically stop codons and corresponding regulatory sequences; if the transferred nucleic acids are only to be transcribed, they are generally poly-A sequences.

According to the invention, vectors refers to plasmids, cosmids, viruses and other current vectors known in the field of gene technology, with which nucleic acid molecules can be transferred to plants or plant cells.

In order to prepare the introduction of foreign genes into higher plants or the cells of higher plants, a large number of cloning vectors are available which contain a replication signal for E. coli and a marker gene for the selection of transformed bacterial cells. Examples of such vectors are pBR322, pUC series, M13 mp series, pACYC184, etc. The required sequence can be introduced into the vector at an appropriate restriction site. The plasmid obtained is used for the transformation of E. coli cells. Transformed E. coli cells are cultivated in an appropriate medium, and finally harvested and lysed, followed by recovery of the plasmid. Methods such as restriction analyses, gel electrophoreses and other biochemical/molecular biological methods are generally used for characterizing the plasmid DNA obtained. Following each manipulation the plasmid DNA can be cleaved and the DNA fragments obtained can be combined with other DNA sequences. Each plasmid DNA sequence can be cloned into the same or other plasmids. Standard cloning methods can be taken from, e.g., Sambrook et al., 2001 (Molecular cloning: A laboratory manual, 3^{rd} edition, Cold Spring Harbor Laboratory Press).

There are a number of well-known techniques available for the introduction of DNA into a plant host cell, and the person skilled in the art can determine the appropriate method in each case. These techniques include the transformation of plant cells with T-DNA by using Agrobacterium tumefaciens or Agrobacterium rhizogenes as a transformation agent, the fusion of protoplasts, the direct gene transfer of isolated DNA into protoplasts, the electroporation of DNA, the introduction of DNA by means of the biolistic method, as well as other techniques. In so doing, both stable and transient transformants can be generated.

With the injection and electroporation of DNA into plant-cells there are no special requirements per se for the plasmids used. The same applies for direct gene transfer. Simple plasmids, such as pUC derivates can be used. If, however, whole plants are to be regenerated from such transformed cells, the presence of a selectable marker gene is necessary. The person skilled in the art is acquainted with the current selection markers, and can select an appropriate marker. Standard selection markers are those which mediate resistance to a biocide or an antibiotic such as kanamycin, G418, bleomycin, hygromycin, methotrexat, glyphosat, streptomycin, sulfonyl urea, gentamycin or phosphinotricin and such, to the transformed plant cell.

Dependent upon the method of introduction of the desired gene into the plant cell, other DNA sequences may be required. For example, if the Ti or Ri plasmid is used for the transformation of the plant cell, at least the right flanking region, often however the right and the left flanking region of the T-DNA contained in the Ti or Ri plasmid must be linked as a flanking region with the gene to be introduced.

If agrobacteria are used for the transformations, the DNA to be introduced must be cloned in special plasmids, either in an intermediary or in a binary vector. Based on sequences that are homologous to sequences in the T-DNA, the intermediary vectors can be integrated into the Ti or Ri plasmid of the agrobacteria by homologous recombination. This plasmid also contains the vir region necessary for the transfer of the T-DNA. Intermediary vectors cannot replicate in agrobacteria. By means of a helper plasmid, the intermediary vector can be transferred to Agrobacterium tumefaciens (conjugation). Binary vectors can replicate in E. coli as well as in agrobacteria. They contain a selection marker gene and a linker or polylinker framed by the right and left T-DNA border regions. They can be transformed directly into the agrobacteria (Holsters et al. (1978), Molecular and General Genetics 163, 181-187). The agrobacterium serving as a host cell should contain a plasmid which carries a vir region, which is necessary for the transfer of the T-DNA into the plant cell. T-DNA can also be present. This type of transformed agrobacterium is used for the transformation of plant cells.

The use of T-DNA for the transformation of plant cells has been intensively investigated and is described in EP 120 515.

For the transfer of DNA into the plant cell, plant explants can be cultivated specifically for this purpose with Agrobacterium tumefaciens or Agrobacterium rhizogenes. From the infected plant material (for example, pieces of leaf, stem segments, roots, but also protoplasts or suspension-cultivated plant cells) whole plants can be regenerated in an appropriate medium which can contain antibiotics or biocides for the selection of transformed cells. The regeneration of the plants takes place according to standard regeneration methods and using the common nutrient solutions. The plants and plant cells obtained in this way can by examined for the presence of the DNA introduced.

The person skilled in the art is acquainted with other possibilities for the introduction of foreign DNA using the biolistic method or by protoplast transformation (see L. Willmitzer (1993) Transgenic Plants in: Biotechnology, A Multi-Volume Comprehensive Treatise (publisher: H. J. Rehm et al.), volume 2, 627-659, VCH Weinheim, Germany).

Whereas the transformation of dicotyledenous plants or their cells by means of Ti plasmid vector systems with the help of Agrobacterium tumefaciens is well established, new work points to the fact that monocotyledonous plants or their cells are also very accessible to transformation by means of vectors based on agrobacteria (see e.g. Chan et al. (1993), Plant Mol. Biol. 22, 491-506).

Alternative systems for the transformation of monocotyledonous plants or their cells are transformation by means of the biolistic approach (Wan and Lemaux (1994) Plant Physiol. 104, 37-48; Vasil et al. (1993) Bio/Technology 11, 1553-1558; Ritala et al. (1994) Plant Mol. Bio. 24, 317-325; Spencer et al. (1990), Theor. Appl. Genet. 79, 625-631), protoplast transformation, electroporation of partially permeabilized cells, as well as the introduction of DNA by means of glass tissues.

The transformed cells grow within the plant in the normal way (see also McCormick et al. (1986), Plant Cell Reports 5, 81-84). The resulting plants can be raised in the normal way and be crossed with plants which have the same transformed genetic disposition or other genetic dispositions. The resulting hybrid individuals have the respective phenotypical properties.

Two or more generations should be raised in order to ensure that the phenotypical feature remains stable and is inherited. Seeds should be harvested as well to ensure that the respective phenotype or other characteristics are maintained.

Similarly, by using standard methods, transgenic lines can be determined which are homozygous for the new nucleic acid molecules and their phenotypical characteristics with regard to a present or non-present pathogen responsiveness is investigated and compared with that from hemizygous lines.

Of course, plant cells that contain the nucleic acid molecules according to the invention and plant cells (including protoplasts, calli, suspension cultures and the like) can be further cultivated.

The vectors described above can be transferred to plant cells in various ways. Whether the vectors are in linear or circular form depends upon the application in question. The person skilled in the art knows whether and when to use respective linearized vectors. For example, the person skilled in the art knows that, for the production of specific knock-outs of genes for DnaJ-like proteins by homologous recombination, it can suffice to linearize the corresponding vectors and inject them into transgenic plants.

According to the invention, the term transgenic plants includes not only the plant in its entirety, but also the plant components in which, according to the invention, the expression of plant DnaJ-like proteins that interact with viral components, is suppressed. These plant components can be plant cells, plant seeds, leaves, blossoms and pollen. According to the invention, “transgenic plant” also means the propagation material from transgenic plants according to the invention, such as seeds, fruits, cuttings, tubers, roots pieces etc., whereby this propagation material, if necessary, contains transgenic plant cells as described above, as well as parts of these plants such as protoplasts, plant cells and calli.

With the production of transgenic plants, there are several different methods and possibilities, as already described above. In general, plants and plant cells can be modified with the help of conventional gene-technological transformation methods in such a way that the new nucleic acid molecules are integrated into the plant genome, i.e. so that stable transformants are produced and the nucleic acid molecules introduced are replicated with the plant genome. Dependent upon the vector system used, it is also possible according to the invention, to produce plants with which the nucleic acids to be transferred are contained in the plant cell or the plant as an independently replicating system. The vectors used for transfer of the plants must accordingly have DNA sequences that allow the replication of plasmids used to transfer within the cell.

Another method according to the invention for the production of transgenic plants with increased virus resistance involves the expression of dominant negative mutants of plant DnaJ-like proteins in the plant or in the plant cells. By introducing dominant negative mutants into the endogenous plant DnaJ-like proteins, non-transgenic plants and plant cells can be produced according to the invention that are equipped with increased virus resistance.

“Dominant negative mutants” refers to plant DnaJ-like proteins according to the invention that have mutations and/or deletions which prevent the plant DnaJ-like proteins from interacting either with the viral components and/or with the other components of the chaperone system.

If these dominant negative mutants are expressed or overexpressed in the transgenic cell or plant, they are able to outcompete the interaction of the viral components with the wild type DnaJ-like proteins or the interaction of the wild type DnaJ-like proteins with the other factors of the chaperone system, so that the virus has no possibility of propagation. Surprisingly, this method allows the production of transgenic plants equipped with increased virus resistance, and at the same time having an essentially normal phenotype, even though such dominant negative mutants should have a negative effect upon the endogenous chaperone system of plant cells.

The person skilled in the art is aware that virus resistant transgenic plants and plant cells according to the invention can be produced not only by the expression or overexpression of dominant negative mutants of plant DnaJ-like proteins, but also by the expression or overexpression of dominant negative mutants of DnaJ-like proteins from other organisms. This can involve DnaJ proteins from prokaryotes such as E. coli or the homologues of the same, and orthologues from eukaryotes such as yeasts (e.g. S. cerevisiae), C. elegans or higher mammals such as mouse, rat and human. It is a prerequisite that the expression of these dominant negative mutants gives rise to competition of the endogenous plant DnaJ-like proteins with viral components and/or their cellular interaction partners. The DnaJ-like proteins from other organisms can be identified by the database analyses and homology comparisons described above. It is a prerequisite that they are equipped with a J domain.

Transgenic plants which express dominant negative mutants from DnaJ-like proteins, and preferably plant DnaJ-like proteins, can be produced by transferring to plant cells a vector which contains the following DNA sequences in 5′-3′-orientation: a promoter which is functional in plants; a DNA sequence operatively linked to the promoter that includes the sequence encoding DnaJ-like proteins, preferably plant DnaJ-like proteins or fragments thereof, whereby the sequence has mutations or deletions that have a negative effect upon the interaction of DnaJ-like proteins with viral components or other factors of the chaperone system; and a termination signal.

The promoters and termination signals can be the same promoters and signals as described above. The same applies to additional regulatory elements, and the type of the vector to be used. The person skilled in the art will choose the corresponding regulatory elements and the vector to correspond to the respective requirements.

The person skilled in the art can identify dominant negative mutations and/or deletions by routine experimentation. A series of mutations in the J domain are well known which e.g. inhibit interaction with the DnaK-like proteins and their homologues. To these belong, in particular, mutations within the three regions encoding helices I to III, in particular the HPD tripeptide (Kelley, 1999, Current Biology, 9, 305-308).

Mutations which specifically prevent the interaction of DnaJ-like proteins, and preferably the plant DnaJ-like proteins, with the viral components can be determined, e.g., by producing recombinant DnaJ-like proteins, preferably plant DnaJ-like proteins, which carry the different mutations and/or deletions, and testing these recombinant proteins in binding assays with viral components. In the same way, dominant negative mutants of plant DnaJ-like proteins can be determined that are no longer able to interact with the cellular binding partners.

In the same way, e.g. in vitro binding tests can be used to test whether dominant negative mutants of DnaJ-like proteins, and preferably plant DnaJ-like proteins, are able to compete with the plant wild type DnaJ-like proteins for interactions with the viral components and/or the cellular binding partners.

Dominant negative mutations means all types of mutation, i.e. insertions, deletions and point mutations, which are able to prevent the interaction of plant DnaJ-like proteins with viral factors and/or cellular binding partners.

With the methods according to the invention for the production of transgenic plants with increased virus resistance by the expression of dominant negative mutants of DnaJ-like proteins, a modulation of the level of DnaJ-like proteins interacting with virus components takes place, with no silencing of the host factors, which gives the additional advantage that the mechanism offers no direct point of attack for virus-encoded suppressors, as occurs with RNAi mediated gene silencing.

For the production of transgenic plants with increased resistance to potyviruses, e.g. dominant negative mutants of the DnaJ-like proteins NtCPIP1 and NtCPIP2a, in which the first 65 or 66 amino acids are missing (NtCPIP1Δ65N and NtCPIP2aΔ66N), can be stably expressed in plant cells. These dominant negative mutants of NtCPIP1 and NtCPIP2a are not equipped with the J domain, but can still interact with the capsid protein (CP) of PVY. These dominant negative mutants compete in their expression with the endogenous WT DnaJ-like proteins for the binding to the PVY-CP and so inhibit the spreading of the virus. The person skilled in the art is acquainted with the expression of recombinant proteins in plant cells and with suitable vectors and plasmids. Analogously, other DnaJ-like proteins such as NtCPIP2b and NtDnaJ-M541, the N terminus of which is deleted, can be used to produce transgenic plants with increased virus resistance. Similarly, for example, the DnaJ-like proteins from A. thaliana mentioned above can be used as dominant negative mutants in which the J domain has been deleted.

The person skilled in the art knows how point mutations, insertion mutations or deletion mutations can be introduced in the coding nucleic acid sequences for DnaJ-like proteins. PCR techniques can, e.g., be favored for the introduction of point mutations (see e.g., “PCR technology: Principle and Applications for DNA Amplification”, H. Ehrlich, id., Stockton Press). Examples for the introduction of point mutations or deletion mutations into DNA sequences encoding proteins can also be found in the examples, below.

Transgenic plants with increased virus resistance can also be produced in such a way, according to the invention, that e.g. a recombinant antibody which specifically blocks or competes the interaction of plant DnaJ-like proteins with the viral components or the factors of the chaperone system can be expressed in the plants. These antibodies can, in particular, be directed against the J domain. The person skilled in the art is acquainted with how such recombinant antibodies against a specific domain of plant DnaJ-like proteins can be isolated and identified, and such information is described in the literature (e.g., Harlow et al., 1999, Using antibodies: a laboratory manual, Cold Spring Harbor Laboratory Press).

According to the invention, recombinant antibodies include the different known forms of recombinant antibodies, as described e.g. in Skerra et al. (Curr. Opin. Immunol. (1993) 2, 250-262). The recombinant antibodies according to the invention include the so-called Fab fragments, Fv fragments, scFv antibodies, scFv homodimers, which are joined together by means of disulfide bonds, and so-called VH chains. The Fab fragments consist of assembled entire light and shortened heavy chains, whereas Fv fragments consist of non-covalently joined VH and VL chains. An overview of the fragments and recombinant antibodies is specified in Conrad et al. (Plant Mol. Biol. (1998) 38, 101-109). The Fab and Fv fragments specified can associate with one another in vivo.

Because this process may not run efficiently, the use of scFv antibodies is favored according to the invention. scFv antibodies consist of the variable region of the light chain and the variable region of the heavy chain fused together by means of a flexible linker peptide. The production of such scFv antibodies has been intensively described in the prior art (see e.g. Conrad et al., vide supra; Breitling et al. (1999) Recombinant Antibodies, John Wiley & Sons, New York). The scFv antibodies exhibit the same antigen specificity and activity as normal antibodies, but need not be assembled in vivo from individual chains in the same way as natural or recombinant antibodies. They are therefore particularly suitable for the methods according to the invention.

The above references describe in detail how nucleic acid sequences which encode the scFv antibodies favored according to the invention can be isolated and produced by the person skilled in the art.

Briefly, one normally starts with existing hybridoma cell lines which produce monoclonal antibodies. cDNAs encoding the light and heavy chains of the antibody are then isolated. The second step is to fuse together the coding regions for the variable region of the light and the heavy chain into one molecule.

Another way of producing recombinant antibodies, with which the person skilled in the art is acquainted, is the screening of libraries of recombinant antibodies (so-called “phage display libraries”, see also Hoogenboom et al. (2000) Immunology Today 21, 371-378; Winter et al. (1994) Annu. Rev. Immunol. 12, 433-455; De Wildt et al. (2000) Nat. Biotechnol. 18, 989-994). By means of procedures known to the person skilled in the art, this method can be used to enrich, select and isolate recombinant antibodies against a specific antigen.

Moreover, according to the invention transgenic plants with increased virus resistance can be produced by the expression of inhibitors that specifically inhibit the interaction of DnaJ-like proteins with viral components or other factors of the cellular chaperone system. These inhibitors can be e.g. peptides which bind in the respective binding pockets of the DnaJ-like proteins that interact with the viral components or other factors of the chaperone system.

Another possibility for the production of transgenic plants with increased virus resistance involves overexpressing just the J domain. In this way, the interaction of viral components with plant DnaJ-like proteins can be outcompeted. Typically, the overexpressed J domain has the SEQ ID NO:7 or is homologous thereto. The overexpressed J domain competes with endogenous plant DnaJ-like proteins for binding partners, so that propagation of the virus is inhibited.

Examples are given below of how plant DnaJ-like proteins which interact with viral components, can be identified, how the binding specificity of these plant DnaJ-like proteins can be determined, and how transgenic plants with increased virus resistance can be produced by silencing of the plant DnaJ-like proteins. These examples are non-exclusive.

Further subject-matter of the present invention involves plant cells and plants in which the endogenous genes of plant DnaJ-like proteins have mutations, i.e. substitutions, insertions and/or deletions, so that the endogenous DnaJ-like proteins expressed are no longer able, or only able under certain circumstances, to interact with viral factors and/or their endogenous cellular binding partners. Plants or plant cells that contain these endogenous gene copies for DnaJ-like proteins showing mutations can be distinguished by increased transient or permanent virus resistance to the above specified virus groups and strains, like the transgenic plants and plant cells described above. Such plants and plant cells which, unlike the plants and plant cells specified above, are not transgenic can be produced by conventional mutagenesis.

According to the invention, these non-transgenic plants or plant cells must, however, exhibit the above-specified types of mutation in the genes encoding plant DnaJ-like proteins, which lead to a modulation of the expression of the plant DnaJ-like proteins and/or the binding characteristics of the plant DnaJ-like proteins. Modulation of the expression of the endogenous plant DnaJ-like proteins can e.g. mean that the expression of endogenous plant DnaJ-like proteins is down-regulated by means of mutations in regulatory DNA elements of the genes of the plant DnaJ-like proteins, such as promoters, enhancers or generally so-called “upstream activating sequences”.

Within the framework of the present invention, the modulation of the binding characteristics of the plant DnaJ-like proteins means that the above-specified types of mutations lead to a change in the binding characteristics of the endogenous plant DnaJ-like proteins to the viral factors and/or the normal cellular binding partners. A modulation of the binding characteristics of the DnaJ-like proteins is preferred, causing them to no longer interact, or to interact only under certain circumstances, with viral factors and/or their cellular partners. A combination of the modulation of the expression and the binding characteristics of the plant DnaJ-like proteins is also possible.

For example, plants or plant cells can have mutations in the gene sequences for DnaJ-like proteins that lead to the reduction of the expression of these proteins. Other plants or plant cells have mutations which lead to the dominant negative mutants described above. In both cases, plants with increased virus resistance are obtained.

The person skilled in the art is aware that, e.g., plants and plant cells can also be produced by mutagenesis which, because of mutations in enhancer and/or promoter sequences of the genes for plant DnaJ-like proteins, show a reduction of the expression of these proteins. Such plants or plant cells can at the same time show mutations in the coding regions of the genes encoding plant DnaJ-like proteins, which cause the remaining expressed plant DnaJ-like proteins to no longer interact, or to interact only to a limited extent, with the viral and/or other cellular binding partners. On the other hand, respective mutations in enhancer and/or promoter sequences and in the coding sequences can have the effect that a dominant negative mutant of plant DnaJ-like proteins, as described above, which is no longer, or only to a very limited extent, able to interact with viral and/or normal cellular interaction partners, is overexpressed, giving rise to the competition reaction described above.

These plants are distinguished by increased transient or permanent virus resistance to the virus classes, groups and strains specified above.

Preferably, the non-transgenic plants and plant cells according to the invention, which are distinguished by a modulation of the expression and/or the binding characteristics of the endogenous plant DnaJ-like proteins and which have permanent or transient virus resistance, are produced by means of the so-called “TILLING” method (Targeting Induced Local Lesion in Genomes). The TILLING method has been described in detail in Colbert et al. (2001, Plant Physiology, 126, 480-484), McCallum et al. (2000, Nat. Biotechnol., 18, 455-457) and McCallum et al. (2000, Plant Physiology, 123, 439-442), each of which are herein incorporated by reference in their entirety.

The TILLING method is a so-called reverse genetics strategy which combines the production of high frequencies of point mutations in mutagenized plant collections, e.g. by means of chemical mutagenesis with ethyl methane sulphonate (EMS), with the fast systematic identification of mutations in target sequences. First, the target sequence is amplified via PCR in DNA pools of mutagenized M2 populations. Denaturation and annealing reactions of the heteroallelic PCR products allow the formation of hetero-duplexes, wherein one DNA strand originates from the mutated and the other from the “wild-type” PCR product. A so-called mismatch then takes place at the site of the point mutation, which can be identified either by means of denaturating HPLC (DHPLC, McCallum et al., 2000, Plant Physiol., 123, 439-442) or with the Cell mismatch detection system (Oleykowsky et al., 1998, Nucl. Acids Res. 26, 4597-4602). Cell is an endonuclease which recognizes the mismatches in heteroduplex DNA and specifically cleaves the DNA at these sites. The cleavage products can then be separated and detected by means of automated sequencing gel electrophoresis (Colbert et al., 2001, vide supra). Following identification of target gene-specific mutations in a pool, individual DNA samples are analyzed accordingly to isolate the plant with the mutation of interest. In this way, the identification of the mutagenized plants or plant cells can be made with the plants and plant cells according to the invention after the production of the mutagenized plant populations by the use of primer sequences targeted at DnaJ-like proteins. The TILLING method is generally applicable for all plants, and so the cultivated and agricultural plants specified above are suitable for the methods according to the invention.

### EXAMPLES

### Example 1

### Identification of Plant DnaJ-Like Proteins which Interact with Viral Components

The following example shows how plant DnaJ-like proteins were identified from Nicotiana tabacum by a two-hybrid screen which interact with the capsid protein (CP) from potato virus Y (PVY) and tobacco etch virus (TEV). In order to give a more concise overview, the methods and materials used within the framework of this experiment will first be discussed.

General Cloning Processes

Cloning processes such as: restriction digest, DNA isolation, agarose gel electrophoresis, purification of DNA fragments, transfer of nucleic acids to nitrocellulose and nylon membranes, linking of DNA fragments, transformation of E. coli cells, culture of bacteria, and sequence analysis of recombinant DNA were carried out as described by Sambrook et al. (vide supra.) The transformation of Agrobacterium tumefaciens was realized according to the method of Hofgen and Willmitzer (Nucl. Acid Res. (1988) 16, 9877). The culture of agrobacteria was performed in YEB medium (Vervliet et al. Gen. Virol. (1975) 26, 33). Work with yeast was carried out according to the protocols in the Yeast Protocols Handbook (1999, Clontech Laboratories, Inc., Palo Alto, Calif., USA).

Bacterial/Yeast Strains and Plasmids

E. coli (XL-1 Blue, XL-MRF′ and XLOLR) bacteria were obtained from Stratagene and Clontech (KC8). The yeast strain Y190 used in work with the yeast two hybrid system was introduced by Harper et al. (1993, Cell 75: 805). The agrobacterium strain used for the plant transformation (C58C1 with the plasmid pGV 3850kan) was described by Deblaire et al. (1985, Nucl. Acid Res. 13: 4777).

For the cloning, the vectors pGEM-T (Promega), pCR-blunt (Invitrogen), pBinAR (Hofgen and Willmitzer 1990, Plant Sci, 66: 221) and the vector pUC-RNAi described in detail below, were used. For the work with the yeast two hybrid system the vectors pGBT9, pGBKT7 (Clontech) and pAD-GAL4 (Stratagene) were used.

Tobacco Transformation

For the transformation of tobacco plants (Nicotiana tabacum L. cv. Samsun NN), 10 ml of an overnight culture of Agrobacterium tumefaciens grown under selection were centrifuged, the supernatant was discarded, and the bacteria were resuspended in the same volume of antibiotic-free medium. In a sterile petri dish, leaf discs from sterile plants (diameter approx. 1 cm) were bathed in this bacterial solution. Afterwards, the leaf discs were placed on MS medium in petri dishes (Murashige and Skoog, Physiol. Plant. (1962) 15, 473) with 2% saccharose and 0.8% bacto-agar. Following 2 days incubation in the dark at 25° C., they were transferred to MS medium with 100 mg/l kanamycin, 500 mg/l claforan, 1 mg/l benzyl amino purine (BAP), 0.2 mg/l naphthyl acetic acid (NAA), 1.6% glucose and 0.8% bacto-agar, and the cultivation was continued (16 hours light/8 hours dark). The growing spouts were transferred to hormone-free MS medium with 2% saccharose, 250 mg/l claforan and 0.8% bacto-agar.

Isolation of a cDNA Encoding Potato Virus Y (PVY) Envelope Protein (Capsid Protein, CP) from Virus-Infected Leaf Material

The cloning of a cDNA encoding PVY capsid protein was performed by means of RT-PCR (Reverse Transcription Polymerase Chain Reaction) of RNA from virus-infected leaf material. For this, leaves from Nicotiana tabacum L. variety Samsum NN were infected with a PVY (N strain, pVy N) field isolate (Bundesanstalt fir Züchtungsforschung an Kulturpflanzen Aschersleben) according to the method described in Herbers et al. (1996, Plant Cell, 8, 793). Total RNA was isolated from the leaf material according to Logemann et al. (1987, Anal. Biochem. 163: 16). Amplification took place using the specific PCR primers D44 (5′-ATGAATTCGCAAATGACACAATTGATGC-3′) and D45 (5′ATGTCGACCATGTTCTTGACTCCAAGTAG-3′) which were derived from a published PVY^{N} sequence (gene bank accession D00441) and provided with EcoRI-(D44) and Sal/I (D45) restriction sites (underlined). 5′ primer D44 includes the bases 8573-8592 and 3′ primer D45 includes the bases 9353-9573.

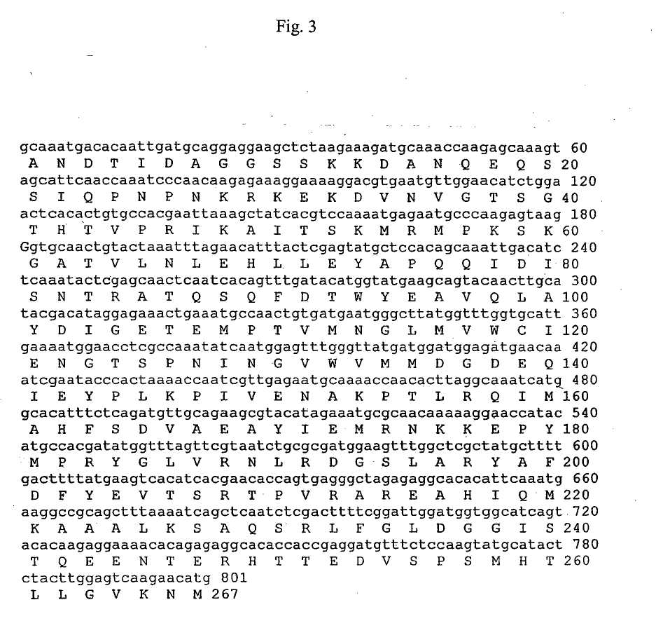

The RT-PCR took place in a coupled reverse transcription/cDNA amplification assay according to the protocol of the Tth DNA polymerase (Biomaster). The reaction mixture (20 μl) for the reverse transcription contained 0.1 μl total RNA as template material, 3.2 μl primer D45 (5 μM), 2 μl Tth polymerase 10× reaction buffer, 0.4 μl dNTPs (10 nM dATP, dCTP, dGTP, dTTP), 2 μl MnCl₂ (10 mM) and 5 U Tth DNA polymerase. The mixture was incubated for 5 min at 60° C., and then for 10 min at 70° C. before Chelating. 5× reaction buffer, 3.2 μl primer D44 (5 μM), and 3 μl MgCl₂ (50 mM) were added to the reaction mixture (final volume 100 μl) for the cDNA amplification. The PCR amplification was realized in a GeneAmp PCR system (Perkin Elmer) with the following program: 1 cycle 95° C. 2 min, 60° C. 1 min; 35 cycles 95° C. 1 min, 60° C. 1 min; 1 cycle 95° C. 1 min, 60° C. 7 min. The cDNA fragment obtained was cloned into the vector pGEM-T (promega) and the identity of the amplified PVY CP cDNA verified by means of sequence analysis (FIG. 3).

Identification of PVY CP Interacting Proteins

PVY CP interacting proteins were identified by means of a yeast two hybrid approach according to conventional techniques, as described in MacDonald (ed.) et al.

Two Hybrid Systems: Methods and Protocols, Methods in Molecular Biology, Vol. 177, Totowa; N.J., USA). The establishing of a yeast two hybrid cDNA library was performed according to the protocol and by using the materials from the “HybriZAP Two-Hybrid cDNA Gigapack Cloning Kit” (Stratagene) and involved the following steps: Total RNA was isolated from “source” leaves from Nicotania tabacum L variety Samsun NN according to the method of Logemann et al. (1987, vide supra). By using Oligo dT cellulose Poly(A)⁺ RNA was obtained from 3.7 mg leaf RNA, according to the method of Sambrook et al. (vide supra).

5 μg poly(A)⁺ RNA was transcribed, by means of reverse transcriptase, in first strand cDNA which served as the template for the second strand synthesis by means of the DNA polymerase. After filling up overhanging DNA ends, EcoRI adapters were added, and were phosphorylated by means of a T4 polynucleotide kinase. After XhoI digestion of the adapters, the cleaved cDNA population was size fractionated by means of a sepharose CL-2b column and ligated into an EcoRI/XhoI cut HybriZAP vector. The ligation mixture was packaged in Lambda Phages with the help of the “Gigapack III Gold Packaging Extract” and after a mass in vivo excision and amplification step, the primary HybriZAP cDNA library converted into a pAD-GAL4 phagemid cDNA library.

The cDNA encoding PVY CP was cloned as EcoRI/SAL/I fragment in an open reading frame with the GAL4 binding domain into the vector pGBT9 (Clontech). This “bait” plasmid construct was first transferred into the yeast reporter strain Y190 (vide supra), and the cDNA library plasmids were transformed therein according to the PEG/LiAc/ssDNA method described in Schiestl and Gietz (1989, Curr. Genet. 156, 339). For the selection of interacting plasmids the yeast cells were plated on minimal medium without the amino acids tryptophan, leucine, histidine (SD Trp⁻/Leu⁻/His⁻) and with 25 mM 3-amino-1,2,4-triazol (3-AT; Gietz et al. 1995, Yeast 11: 355; Bartel and Field 1995, Methods in Enzymology 254: 241).

The transformation efficiency was determined by the plating of aliquots on SD Trp⁻/Leu⁻ plates with 8×10⁷ screened transformants. Following 7-10 days incubation at 30° C., 33 yeast colonies with clear growth (at least 2 mm in diameter) were isolated and tested for expression of the lacZ reporter gene according to the beta galactosidase filterlift assay method described by Breeden and Naysmyth (1989, Cold Spring Harbor Symposium Quant. Biol. 50: 643). 4 yeast colonies which showed not only growth in the absence of histidine, but also lacZ activity, were selected for further characterization. Total DNA was isolated from the yeast cells and selected, following electroporation in the E. coli strain KC8 (Clontech), for the presence of the pAD-GAL4-cDNA plasmids.

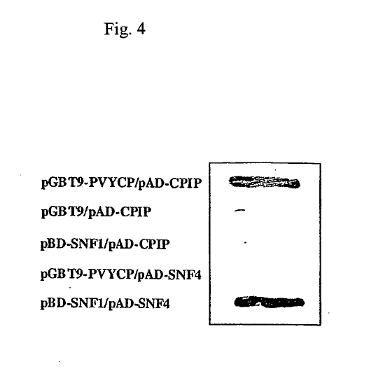

The specificity of the interaction was verified by means of co-transformation of the activation domain plasmids and the bait plasmids with the unrelated control plasmids pBD-p53, pBD-SNF1 and pAD-SNF1 (Stratagene) (FIG. 4). The isolated activation domain plasmids showed specific interaction with the PVY CP construct and not with the controls pBD-p53 and pBD-SNF1, so that the activation domain-fused cDNAs were sequenced. The four clones (termed Capsid protein interacting protein, CPIP7, 13, 19, 29) were identical to one another, whereby CPIP29 encoded an additional 25 amino acids at the N terminus. Following the screening of a λ ZAP II cDNA library from tobacco leaf material (Herbers et al. 1995, Plant Mol. Bio. 29: 1027), the whole cDNA was isolated with CPIP7 as a cDNA probe, whereby standard protocols from Sambrook et al. (vide supra) were applied.

The full-length cDNA clone was termed NtCPIP1 and coded for 306 amino acids which, within the first 70 amino acids, had significant homology to members of the family of DnaJ-like proteins and/or HSP40 heat shock proteins, and included the eponymous J domain (FIG. 2a). The highest homologies (determined according to the Clustal method in the DNA Star package, DNASTAR Inc., Madison, Wis.) were shown by Arabidopsis thaliana clones with 60.8% (Gene Bank Accession AC007258), 60.1% (Gene Bank Accession AC005489) and 58.8% (Gene Bank Accession AC010871) identity on the protein level. Moreover, 56.5% identity to a DnaJ-like protein from Nicotiana tabacum (NtDnaJ_M541; Gene Bank Accession AAF05720) and 55.6% to its orthologue AtA39 (Gene Bank Accession AL021749) from Arabidopsis were detectable, which were identified as plant interaction partners of the TSWV NSm cell to cell transport protein from Soellick et al (2000, Proc. Natl. Acad. Sci. U.S.A. 97, 2373).

With the following screening of a λ ZAP II cDNA library from tobacco leaf material (Herbers et al., 1995, Plant. Mol. Biol., 29: 1027) with CPIP7 as the cDNA probe and using standard methods according to Sambrook et al. (vide supra), two additional DnaJ-like proteins could be identified, the DNA sequences of which are given in FIGS. 2b and 2c along with the derived amino acid sequences.

### Example 2

### Characterization of the Binding Specificity of NtCPIP1

In order to characterize the binding specificity of NtCPIP1, NtCPIP2a and NtCPIP2b, additional potyviral capsid proteins were tested for interaction with NtCPIP1, NtCPIP2a and NtCPIP2b in the yeast two hybrid system. The sequences encoding the capsid proteins of the Tobacco etch virus (TEV, Gene Bank Accession M15239, bases 8518-9309), Tobacco vein mottling virus (TVMV, Gene Bank Accession X04083, bases 2759-3024), Turnip mosaic virus (TUMV, Gene Bank Accession D10601, bases 4027-4890) and Potato virus A (PVA strain DAT, Gene Bank Accession AJ2963 11, bases 8532-9338) were ligated with the GAL4 binding domain in the pGBT9 or pGBKT7 (Clontech) vector in an open reading frame, and the plasmids obtained were co-transformed with pAD-CPIP29 (plasmid deriving from the primary yeast-two-hybrid screen) into the reporter strain Y190.

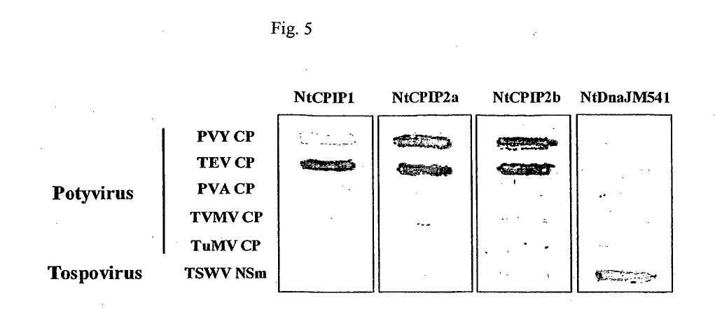

The yeasts were selected for 3-4 days on SD Trp⁻/Leu⁻ medium and tested for expression of the HIS3 and lacZ reporter genes following transfer to SD Trp⁻/Leu⁻/His⁻ medium. It was shown that NtCPIP1, NtCPIP2a and NtCPIP2b interact specifically with the capsid proteins of PVY and TEV, but do not interact with those of TVMV, TuMV and PVA (table 1 and FIG. 5). The specificity of the binding was also confirmed in that there was no detectable binding between NtCPIP1 and the TSWV NSm protein (Soellick et al. 2000, vide supra), in the same way as its interaction partner NtDnaJ-M541 showed no interaction with the PVY and TEV capsid proteins in the yeast two hybrid system (table 1 and FIG. 5).

TABLE 1

| DNA | Activation domain | | | |
|---|---|---|---|---|
| binding domain | NtCPIP1 | NtCPIP2a | NtCPIP2b | NtDnaJ M541* |
| PVY CP | + | + | + | − |
| TEV CP | + | + | + | − |
| PVA CP | − | − | − | − |
| TVMV CP | − | − | − | − |
| TuMV CP | − | − | − | − |
| NSm* | − | − | − | + |

*(Soellick et al. 2000, vide supra)

### Example 3

### Detailed Characterization of the Interaction of PVY CP with NtCPIP1

In the following experiment it was investigated which region of PVY CP is responsible for the interaction with NtCPIP1. For this, a series of N- or C-terminal deleted fragments of CP and a series of CP point mutants were produced as GAL4 binding domain fusion proteins.

Generation of Point Mutants and Deletion Mutants for the Two Hybrid Analysis

Single and double amino acid substitution mutations were introduced in the coding sequence of the PVY CP core region by means of site directed mutagenesis. The “Quick change site-directed mutagenesis” kit (Stratagene) and respective oligonucleotides were also used according to the manufacturer's instructions. For the production of the single amino acid substitution mutants S125W, R157D and D201R, the pGEM/T-PVY CP plasmid was used as template. Double amino acid substitution mutants of PVY CP were obtained by introducing the S125W or D201R mutation into the plasmid which contained the single mutants R157D, or by introducing a D201R mutation into the plasmid which contained the S125W mutant. After the mutation was verified by sequencing, the mutagenized CP sequences were cut out of the pGEMIT vector and cloned into the pGBT9 “bait” vector.

The Δ29N and Δ18C deletion mutants were obtained by PCR amplification of a region encoding CP, which was either lacking the amino acids 1-29 (nucleotides (nt) 88-801 of the PVY CP cDNA sequence) or 249-267 (nt 1-747). The PCR fragments were subcloned into the pCR blunt (in vitro gene) and ligated into the EcoRI/SalI restriction sites of pGBT9.

Binding Specificity of NtCPIP1 and NtCPIP2a for Point Mutants and Deletion Mutants of PVY CP

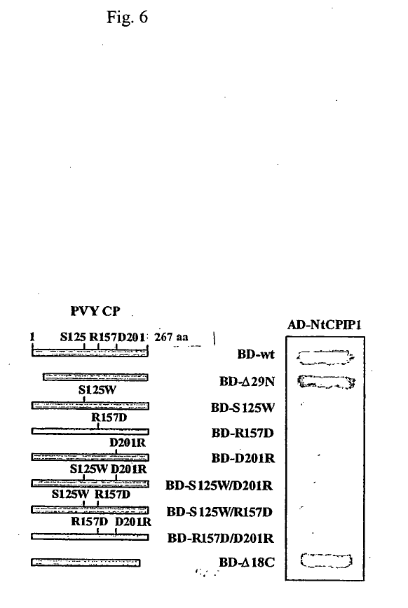

In order to determine the exact binding regions in PVY CP and in NtCPIP1 and NtCPIP2a, the aforementioned point and deletion mutants were tested in the yeast “two hybrid” assay, as already described above for the interaction of different viral CPs with different plant DnaJ-like proteins. As one can see in FIG. 6, the binding of PVY CP to NtCPIP1 is not affected by the N and C terminal deletions of CP. The expression of the lacZ reporter gene was completely prevented by the three point mutants and all double mutants, so that the core region of the CP takes on an essential role in the interaction with NtCPIP 1.

### Example 4

### Production of Virus Resistant Tobacco Plants by the Silencing of NtCPIP1

In order to silence tobacco plants for NtCPIP1, thus making them resistant e.g. to PVY, a plasmid construct was produced, the transcription of which in the cell leads to a double-stranded RNA molecule with a sequence which includes parts of the sequence encoding NtCPIP1. This type of double-stranded RNA molecule should then induce the PTGS system and suppress the expression of NtCPIP1.

Generation of the Plasmid pBinNtCPIP1-RNAi

The in vivo significance of the interaction between PVY CP and NtCPIP 1 during the course of infection should be analyzed by suppression of the NtCPIP1 gene expression in transgenic plants. For this, the strategy of the so-called RNAi “silencing” was followed which is characterized by a high level of suppression frequency and efficiency (Smith et al. 2000, Nature 407:319; Wesley et al. 2001, Plant J 27: 581). In order to produce an RNAi construct for NtCPIP1, first of all the first intron of the GA20 oxidase from Solanum tuberosum (StGA20oxIN, unpublished) was amplified via PCR using the primers GAIN-1 (5′CCT GCA GGC TCG AGA CTA GTA GAT CTG GTA CGG ACC GTA CTA CTC TA-3′) and GAIN-2 (5′-CCT GCA GGG TCG ACT CTA GAG GAT CCC CTA TAT AAT TTA AGT GGA AAA-3′), so that the restriction sites PstI/Sbf1-XhoI-SpeI-BglII were added at the 5′ end, and the restriction sites BamHI-Xba1-SalI-PstI/SbfI at the 3′ end. This PCR fragment was subcloned into a pCR blunt vector and, following a Stul digestion “blunt end”, ligated into a pUC18 vector which was previously opened by a EcoRI/HindIII digestion and filled up by pfu polymerase.

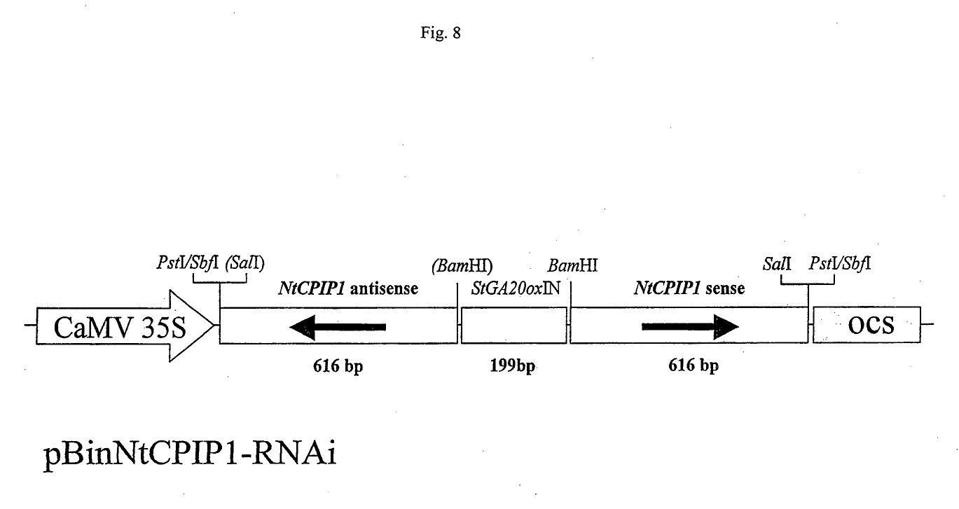

The vector obtained was termed pUC RNAi (FIG. 7). Then a BamHI/SalI linked NtCPIP1 fragment (bases 721-1336) was amplified by PCR, using the primers D153 (5′-GGA TCC CAG AAA ATC TCA TTA GTA GAT GC-3′) and D154 (5′-GTC GAC ATT CAT TAC CAG TCT ACC ACA GC-3′), and following subcloning into the pCR blunt vector, this was inserted successively in sense (s) and antisense (α) orientation, first of all into the BamHI/SalI cut, and then into the BglII/XhoI cut pUC-RNAi. The whole RNAi cassette consisting of αNtCPIP1-StGA20oxIN-sNtCPIP1 was finally ligated into a SbfI cut BinAR via PstI from the pUC RNAi vector, and the plasmid pBinNtCPIP 1-RNAi was thus obtained (FIG. 8). For the plant transformation, the construct was finally transformed into agrobacteria.

Analysis of Transgenic Tobacco Plants for RNAi “Silencing” of NtCPIP1

Tobacco plants (Nicotiana tabacum L. variety Samsun NN) were transformed with pBinNtCPIP1-RNAi carrying agrobacteria according to the method described (vide supra). 81 kanamycin-resistant primary transformants were tested for expression of the construct, first of all using Northern analysis, whereby the NtCPIP1 fragment (BamHI/SalI) used to produce the pBinNtCPIP1-RNAi construct served as a cDNA probe for the RNA hybridization.

Analysis of the Transcription Using “Northern Blot” Analysis

Total RNA was isolated from leaf material, as described (Logemann (1987), Anal. Biochem. 163, 16-20). For the “Northern blot” analysis, 30 μg rRNA was separated on a 1.5% (w/v) formaldehyde agarose gel and blotted onto a GeneScreen (NEN, Boston, Ma). The radioactive labeling of the cDNA was carried out with the help of the “High Prime” kit (Boehringer Mannheim, Germany) and [β-³²P]-dCTP. The hybridization was carried out in the established way (Herbers et al., vide supra), and signals were detected by exposure of Kodak X-ray films (Sigma) or with the help of an “imaging analyzer” (Fuji Bas 2000, Fuji, Tokyo, Japan).

24 plants showed a clear hybridization signal which, however, showed a characteristic “smear” over the RNA lane.

Analysis of Transgenic Plants with RT-PCR

For this reason, the “silencing” was analyzed by semi-quantitative RT-PCR. 20 μg total RNA from selected lines (NtCPIP1-RNAi-2, -3, -18, -69, -79) and from two non-transgenic controls (WT) was first of all digested with DNase (Bohringer Mannheim) at 37° C. for 45 min, and this was then inhibited for 10 min at 65° C. Following phenol/choroform/isoamyl alcohol (25:24:1) treatment, the RNA was precipitated with sodium acetate, washed with 70% ethanol and dissolved in 100 μl DEPC-treated H₂O. The cDNA first strand synthesis was carried out in a mixture with 12.5 μl DNase-treated RNA, 5 μl 5× reaction buffer, 2 μl dNTPs (2.5 mM), 1 μl oligo dT primer (50 mM, dT_{[30]} V[G/C/A]) and 2.5 μl DEPC-treated H₂O following incubation for 5 min at 65° C., then for 5 min at 37° C., and finally after adding 1 μl reverse transcriptase (Moloney Murine Leukemia Virus Reverse Transcriptase, Rnase H minus, M-MLV [H-], Promega) and 1 μl RNAse inhibitor at 37° C. (60 min). Following heat inactivation for 5 min at 95° C., the cDNA was finally added as template for the following PCR.

NtCPIP1 cDNA was amplified with the 5′primer D172 (5′-GGA TCC TCA CGC AAT GCG AGT GAA GAA G-3′) and the 3′ primer D154 (5′-GTC GAC ATT CAT TAC CAG TCT ACC ACA GC-3′), and the internal standard actin with the primer pair D202 (5′-ATG GCA GAC GGT GAG GAT ATT CA-3′) and D203 (5′-GCC TTT GCA ATC CAC ATC TGT TG-3′) (as with AC1 and AC2, Romeis et al. 2001, EMBO J. 20: 5556). The PCR mixtures (total volume 100 μl) were made up as follows: 70 μl H₂O, 5 μl 5′ primer (5 μM), 5 μl 3′ primer (5 μM), 8 μl dNTPs (2.5 mM), 10 μl 10× reaction buffer, 1 μl cDNA and 5 U rTaq DNA polymerase (Takara Shouzo, Japan.) Before the beginning of the amplification cycles, the mixtures were heated for 5 min to 95° C. The polymerization steps were carried out in an automatic T3 thermocycler (Biometra) according to the following program: denaturation 95° C. (1 min), annealing of the primers at 55° C. (45 seconds), polymerase reaction at 72° C. (2 min). 10 μl of the PCR mixture were applied to a gel after 25, 30 and 45 cycles respectively.

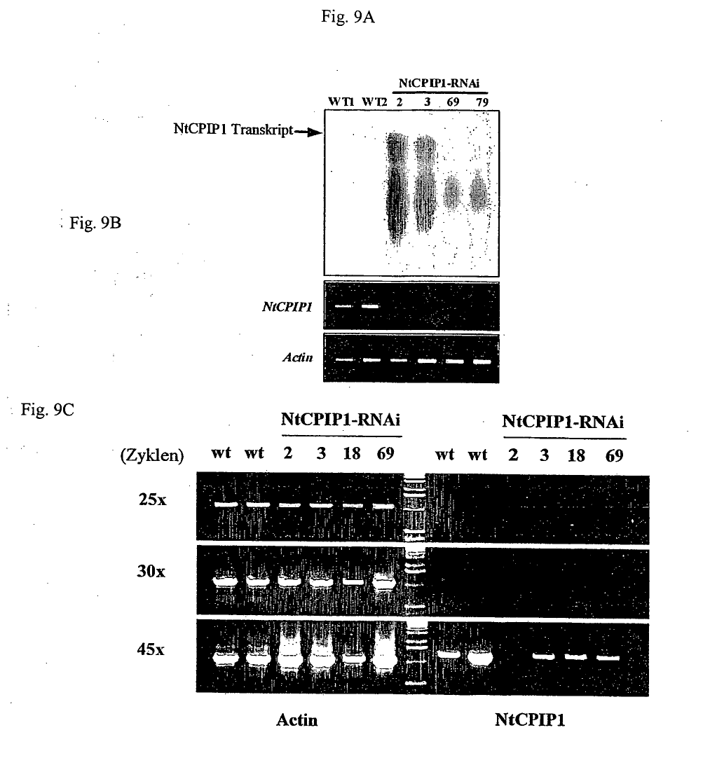

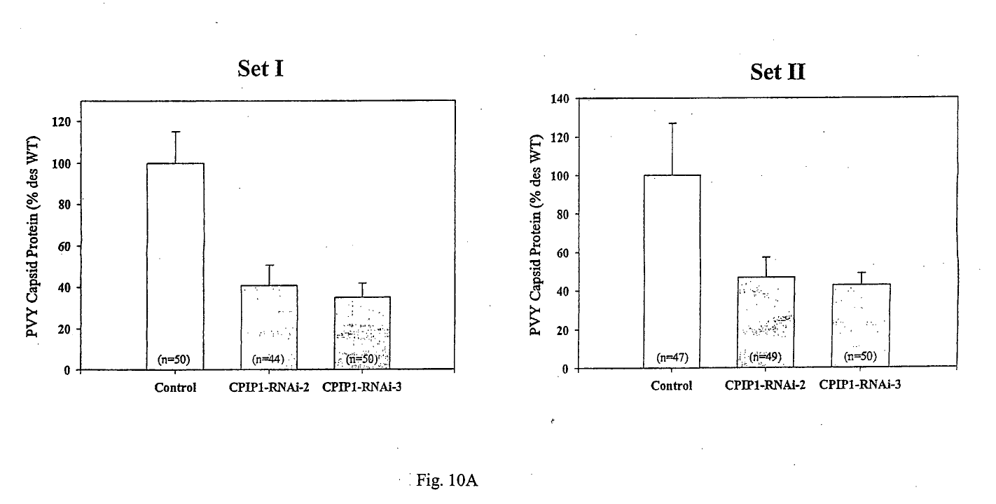

In the non-saturated PCR range (30 cycles) and using the NtCPIP1-specific primer, the result only shows the amplification of products in the wild type controls, and not in the 4 transgenic lines (FIG. 9c). A clear accumulation in lines 3, 28 and 69 can only be identified in the unsaturated range (45 cycles), whereas with line 2, hardly any DNA fragments can be detected, which points to very efficient “silencing”. However, the PCR with the actin-specific primers shows equal DNA bands in the unsaturated range of 25 cycles which proves the application of comparable quantities of the template used (FIG. 9c).

Another transgenic line (79) also showed no amplification of the product in the unsaturated range (30 cycles) (FIG. 9b).

Four of the lines specified (NtCPIP1-RNAi-2, 3, 69 and 79) were also investigated in the Northern blot for expression of NtCPIP1. As shown by FIG. 9a, the transgenic lines showed no expression of this plant DnaJ-like protein.

All of the transgenic lines examined were not phenotypically distinguishable from non-transformed wild plant types (WT). Neither were they distinguishable from transgenic kanamycin-resistant control plants which expressed the B-glucuronidase gene (GUS) under the control of a “source leaf”-specific promoter (line ME-4, Ebneth, M. (1996) doctoral thesis, Freie Universitat Berlin, Germany; data not shown).

PVY Infection of Transgenic Tobacco Plants

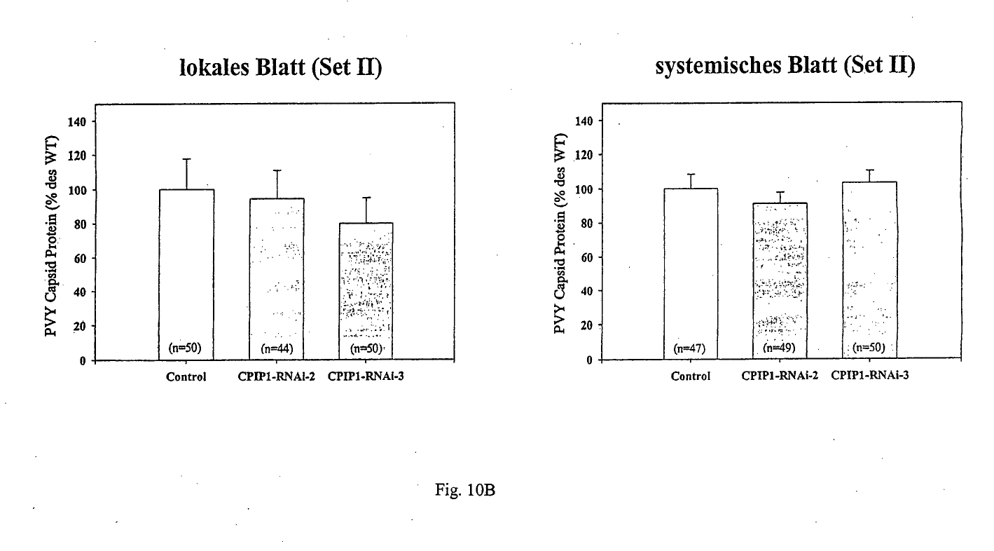

The infection of NtCPIP1-RNAi transgenic lines and non-transformed controls (WT) with PVY^{N} was performed according to the method described in Herbers et al. 1996 (vide supra). The results are shown in FIGS. 10a and 10b. It was shown that a reproducible, significantly increased virus resistance was established in the transgenic lines NECPIR-RNAi-2 and -3 in the locally infected leaf four days post infection (FIG. 10a). This was a transient resistance because six days after the infection, a reduction of the virus titer was no longer detectable (FIG. 10b).

In order to examine the effects of the reduced expression of endogenous NtCPIP1 upon the PVY accumulation and the systemic spreading, the kanamycin-resistant T1 progeny of the selected transgenic plants were infected with PVY^{N} and compared with control lines (WT, ME-4). In order to exclude positional effects in the greenhouse which could arise because of the large number of plants being used, two independent infection experiments were carried out in parallel. The transgenic lines NtCPIP1-RNAi-2 (n=49) and NtCPIP1-RNAi-3 (n=50) were compared to SNN wild plant types (n=47), whereas NtCPIP1-RNAi-69 (n=25) and NtCPIP1-RNAi-79 (n=25) were analyzed in comparison with the transgenic control ME-4 (n=23).

Among the different sets of plants, the transgenic lines and the respective controls were mixed up randomly and the PVY CP protein level was tested four days after mechanical infection in local (inoculated) leaves. The analysis was also carried out six days post infection in local and systemic leaves.

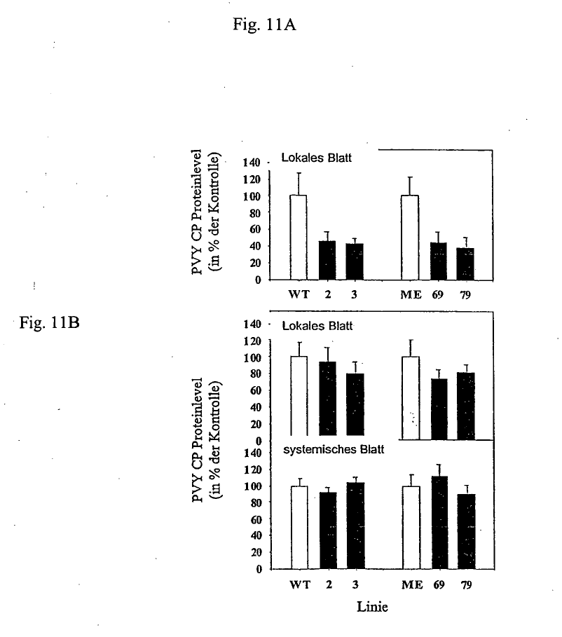

The results are shown in FIGS. 10a and 10b, as well as in 11a and 11b, whereby the values for the WT plants and NtCPIP1-RNAi-2 and 3 lines in FIGS. 11a and b correspond to those in FIGS. 10a and b.

As shown by FIGS. 10a, 10b, 11a and 11b, the transgenic lines NtCPIP1-RNAi-2 and 3 showed a reproducible, significantly increased virus resistance in the locally infected leaf after infection. This was manifested by the fact that, four days after the PVY infection, the virus multiplication was significantly reduced (in comparison with WT plants) by 53.4% and 57.5% in inoculated leaves of NtCPIP1-RNAi-2 and NtCPIP1-RNAi-3. In the local leaves of the transgenic plants NtCPIP1-RNAi-69 and NtCPIP1-RNAi-79, the multiplication of the virus was consistently reduced by 56.0% and 60.1% in comparison with the transgenic control ME-4.

In this case, the resistance was transient because the reduction of the virus titer was no longer as pronounced six days post infection. A comparably strong reduction of the virus level by 59.3% and 65.2% was demonstrated for the transgenic lines NtCPIP1-RNAi-2 (n=44) and NtCPIP1-RNAi-3 (n=50) in comparison to the transgenic control ME-4 (n=50) (data not shown).

The results clearly show that the interaction of the plant DnaJ-like protein NtCPIP1 with the PVY capsid protein plays a role in establishing infection in planta and confirm the two-hybrid data.

The results also show that plants with increased virus resistance can be produced by the silencing of plant DnaJ-like proteins.

Production of Transgenic Tobacco Plants with Multiply Silenced DnaJ-Like Proteins

In the case described above, the occurrence of transient virus resistance can be explained by the fact that there are two additional isoforms of NtCPIP1 in tobacco. These are those also identified as NtCPIP2a and NtCPIP2b within the framework of the present invention. These proteins are possibly not repressed by the RNAi construct used, and can possibly compensate for the RNA silencing of NtCPIP1 because they have been proven to be able to interact with PVY CP (see above).

In the following, therefore, constructs were produced, analogously to the procedure described above, which permit the silencing of NtCPIP2a and NtCPIP2b. With these constructs, respective transgenic plants were produced which were also silenced for NtCPIP1. The plants were examined by semi-quantitative RT-PCR with regard to the NtCPIP1, NtCPIP2a and NtCPIP2b expression. All three proteins were significantly silenced. The transgenic plants were then infected with PVY^{N} and analyzed, as described above. It was shown that the transgenic plants show an effective and lasting virus resistance.

### Example 5

### Production of Virus Resistant Tobacco Plants by Means of Expression of Dominant Negative Mutants of NtCPIP1

The local and transient reduction of the PVY amounts in NtCPIP1—RNAi transgenic plants can be explained by a number of reasons. The most likely reason is that the suppression of NtCPIP1 can be compensated by isoforms which are also able to interact with PVY CP. As already shown above, both NtCPIP2a and NtCPIP2b interact with PVY CP and TEV CP in the yeast “two hybrid” system.

In order to produce permanently virus-resistant plants, shortened dominant negative mutants of the CP interacting plant DnaJ-like proteins were therefore expressed in transgenic plants.

Production of Transgenic Plants which Express Dominant Negative Mutants of NtCPIP1 and NtCPIP2a

A series of N terminal deletion mutants from NtCPIP1 and NtCPIP2a were obtained by PCR amplification using respective oligonucleotides. Fragments which had the sequences for the EcoRI and SalI restriction sites at their 5′- and 3′-ends and which were lacking the amino acid residues 1-65 (Δ65N, nt 246-978), 1-90 (Δ90N, nt 331-987), 1-115 (Δ115N, nt 406-987) of NtCPIP1 and the amino acid residues 1-66 (Δ66N, nt 199-918), 1-94(Δ94N, nt283-918) and 1-119(Δ119N, nt357-918) of NtCPIP2a, were subcloned into the pCR blunt and finally introduced into the pAD-GAL4 “activation domain” vector (Stratagene)

For the ectopic expression of dominant negative mutants of NtCPIP1 and NtCPIP2a, truncated cDNA fragments which did not encode the amino acid residues 1-65 (NtcPIP1 Δ65N) and 1-66 (NtCPIP2a Δ66N) were amplified by PCR. For this the gene-specific primers D249 (5′-GGATCCTATATACGGCGATGAGGCGTTGAAATC-3′) and D251 (5′-GTCGACTTAGTCAACAGTCCTGCCCAGCAC-3′) for NtCPIP1Δ65N as well as D250 (5′-GGATCCTGACGTGTACGGTGATGATGCATTG-3′) and D210 (5′-GTCGACTTAGTCAGCGCTCCTGCACAGTAC-3′) for NtCPIP2aΔ66N were used. Following subcloning into the pCR blunt, the fragments were inserted into the pBinAR between the CaMV 35S promoter and the ocs terminator. In order to improve translation efficiency, the 5′ untranslated “overdrive” sequence (Ω) of the TMV virus Ul (Gallie et al. (1987) Nucl. Acids Res., 15, 8693-8711) was inserted between the promoter and the NtCPIP1Δ65N or NtCPIP2aΔ66N coding sequences. In so doing, an ATG start codon in an optimized plant consensus sequence was also generated within an NcoI cloning site.

In order to transform tobacco plants (Nicotiana tabacum L. cv. Samsum NN), the binary constructs were introduced into the Agrobacterium tumefaciens strain C58C1:pGV2260. An Agrobacterium-mediated gene transfer was then carried out, as described above and in Rosahl et al. (1987, EMBO J., 6, 1155-1159).

Analysis of Transgenic Plants which Express Dominant Negative Mutants of NtCPIP1 and 2a

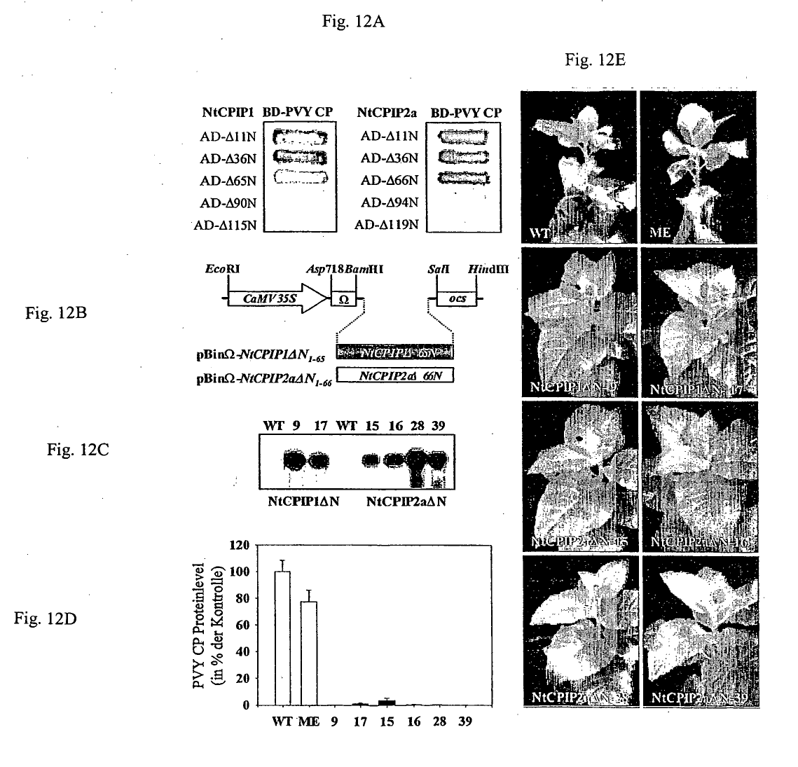

The above-specified N terminal deletion mutants of NtCPIP1 and NtCPIP2a were no longer equipped with the J domain. In the yeast “two hybrid” system, it should therefore first of all be investigated whether the deletion of the main part of the J domain affects the interaction of NtCPIP1 and NtCPIP2a with PVY CP. The N terminal deletion of the amino acids 1-65 and 1-66 of NtCPIP1 and 2a did not, however, lead to a loss of the lacZ reporter gene activity. It can, therefore, be assumed that the J domain is not necessary for the binding to PVY CP. If, however, the N terminal deletions included more than the first 90 amino acids, a total loss of the binding of the NtCPIP isoforms to PVY CP was observed (see FIG. 12a).

Binary constructs were therefore produced (pBinΩ-NtCPIP1 ΔN₁₋₆₅ and pBinΩ-NtCPIP2a ΔN₁₋₆₆, see FIG. 12b) which encoded NtCPIP variants, which had no J domains, but are able to interact with PVY CP. Because of the lack of a J domain, these plant DnaJ-like proteins, which normally act as co-chaperones and regulators of the HSP70 proteins (see above), are no longer able to interact with their endogenous cellular interaction partners.

Following the Agrobacterium-mediated transformation, 26 primary transformants for each construct were cultivated in the greenhouse and examined by means of Northern blotting with regard to the expression of the transgene. For this, the Northern blots were produced as described above. In this way, some plants could be identified which expressed significant quantities of the respective transcripts. Two plants which expressed the construct pBinΩ-NtCPIP1 ΔN₁₋₆₅ (termed NtCPIP1 ΔN-9 and -17) and four transgenic plants which expressed the pBinΩ-NtCPIP2a ΔN_{1*66} (termed NtCPIP1 ΔN-15, -16, -28 and -39) were further analyzed (see FIG. 12c).

1. A method for the production of transgenic plants with increased virus resistance comprising substantially preventing the expression of a plant DnaJ-like protein that interacts with a viral component.

2. The method of claim 1, wherein the expression of the plant DnaJ-like protein is substantially prevented by transferring a nucleic acid molecule to a plant cell, wherein the sequence of said nucleic acid molecule is identical, homologous or complementary to the sequence encoding the plant DnaJ-like protein or a fragment thereof.

3. The method of claim 2, wherein the transferred nucleic acid molecule comprises a sequence that is identical, homologous, or complementary to a sequence encoding the J-domain of the plant DnaJ-like protein or a fragment thereof.

4. The method of claim 2, wherein the transferred nucleic acid molecule comprises a sequence that is identical, homologous or complementary to a sequence encoding a protein specific region of the plant DnaJ-like protein which does not coincide with the J-domain, or a fragment thereof.

5. The method of claim 2, wherein the transferred nucleic acid sequence that is identical, homologous or complementary to the sequence encoding the plant DnaJ-like protein or a fragment thereof, comprises between about 20 to about 1000 nucleotides.

6. The method of claim 2, wherein the transferred nucleic acid sequence is at least 50% homologous to the sequence encoding the plant DnaJ-like protein or a fragment thereof.

7. The method of claim 2, wherein the transferred nucleic acid sequence is at least 50% complementary to the sequence encoding the plant DnaJ-like protein or a fragment thereof.

8. The method of claim 2 comprising:
a) producing a vector comprising the nucleic acid molecule, said vector comprising the following sequence elements in 5′-3′ orientation:
a promoter that is functional in plants;
operatively linked to said promoter, an identical or homologous antisense sequence of the sequence encoding the plant DnaJ-like protein or a fragment thereof, wherein the antisense sequence exhibits a 3′exon sequence at its 3′end that is recognizable by the spliceosome;
an intron;
the identical or homologous sense sequence of the sequence encoding the plant DnaJ-like protein or a fragment thereof, wherein the sense sequence exhibits a 5′exon sequence at its 5′end that is recognizable by the spliceosome;
a termination sequence; and
b) transferring the vector to the plant cell.

9. The method of claim 2 comprising:
a) producing a vector comprising the nucleic acid molecule, said vector comprising the following sequence elements in 5′-3′ orientation:
a promoter that is functional in plants;
operatively linked to said promoter, the identical or homologous antisense sequence of the sequence encoding a plant DnaJ-like protein or a fragment thereof;
a termination sequence; and
b) transferring the vector to the plant cell.

10. The method of claim 9, wherein the vector comprises a regulatory sequence and a functional sequence.

11. The method of claim 10, wherein the regulatory sequence is an enhancer, a replication signal, a selection marker or a sequence allowing for a propagation of the vector in bacteria or replication of the vector in a plant cell.

12. The method of claim 10, wherein the vector is a plasmid, a cosmid or a recombinant virus.

13. The method of claim 12, wherein the vector is pBR322, a pUC vector, an M13 mp vector, or a vector derived from the Agrobacterium Ti or Ri plasmid.

14. The method of claim 9, wherein the promoter is a constitutive promoter selected from the group consisting of: the 35S promoter, the actin promoter, the ubiquitin promoter, a tissue-specific promoter, the phosphoenolpyruvate carboxylase promoter, the fructose-1,6-bisphosphatase promoter, a developmental-specific promoter, a light-induced promoter, a wound-induced promoter and a pathogen-induced promoter.

15. The method of claim 9, wherein the vector is transferred to the plant by transformation, transfection, injection, a biolistic method or electroporation.

16. The method of claim 2, wherein the plant DnaJ-like protein contains a J-domain which is at least 40% homologous, at least 50% homologous, at least 60% homologous, at least 70% homologous, at least 80% homologous, or at least 90% homologous to the amino acid sequence SEQ ID NO. 7.

17. The method of claim 2, wherein the plant DnaJ-like protein is encoded by SEQ ID NO. 1, SEQ ID NO. 2 or SEQ ID NO. 3.

18. The method claim 2, wherein the plant DnaJ-like protein is at least 40% homologous, at least 50% homologous, at least 60% homologous, at least 70% homologous, at least 80% homologous, or at least 90% homologous to SEQ ID NO. 4, SEQ ID NO. 5 or SEQ ID NO. 6.

19. The method claim 2, wherein the transgenic plant exhibit increased resistance to Poty virus, Carla virus, Hordei virus, Potex virus, Bunyai virus, Clostero virus, Cucumo virus, Diantho virus, Tobamo virus, Gemini virus, Lutero virus, Rymo virus, Tobra virus, Furo virus, Como virus, Nepo virus, Bromo virus or Tospo virus.

20. The method of claim 24, wherein the plant exhibits increased resistance to PVY, TEV or TSWV.

21. The method claim 2, wherein the transgenic plant is a monocotyledonous plant selected from the group consisting of: Avena (oat), Triticum (wheat), Secale (rye), Hordeum (barley), Oryza (rice), Panicum, Pennisetum, Setaria, Sorghum (millet), and Zea (maize).

22. The method of claim 2, wherein the transgenic plant is a dicotyledonous plant selected from the group consisting of: cotton, a leguminous plant, soy bean, grape, tomato, sugar beet, potato, an ornamental plant, tobacco and a tree.

23. The method of claim 2 comprising:
a) producing a vector comprising the nucleic acid molecule, said vector comprising the following sequence elements in 5′-3′ orientation:
a promoter that is functional in plants;
operatively linked to said promoter, the identical or homologous sense sequence of the sequence encoding a plant DnaJ-like protein or a fragment thereof, wherein the sense sequence has a self-complementary region;
a termination sequence; and
b) transferring the vector to the plant cell.

24. The method of claim 2 comprising:
a) producing a vector comprising the nucleic acid molecule, said vector comprising the following sequence elements in 5′-3′ orientation:
a promoter that is functional in plants;
operatively linked to said promoter, a DNA sequence that is complementary to the sequence encoding the mRNA of the plant DnaJ-like protein or a fragment thereof;
a DNA sequence encoding ribonuclease P;
a termination sequence; and
b) transferring the vector to the plant cell.

25. The method of claim 2 comprising:
a) producing a vector comprising the nucleic acid molecule, said vector comprising the following sequence elements in 5′-3′ orientation:
a promoter that is functional in plants;
a DNA sequence which is identical or homologous to the sequence encoding the 5′end of the plant DnaJ-like protein;
a DNA sequence encoding a resistance gene;
a DNA sequence which is identical or homologous to the sequence encoding the 3′end of the plant DnaJ-like protein;
a termination sequence;
b) transferring the vector to the plant cell; and
c) allowing the vector to integrate into the plant genome.

26. The method of claim 2 comprising:
a) producing a vector comprising the nucleic acid molecule, said vector comprising the following sequence elements in 5′-3′ orientation:
a promoter that is functional in plants;
operatively linked to said promoter, a DNA sequence encoding a ribozyme that specifically recognizes the mRNA of a plant DnaJ-like protein;
a termination sequence; and
b) transferring the vector to the plant cell.

27. A method for the production of a transgenic plant with increased virus resistance comprising:
a) producing a vector which comprises the following elements in 5′-3′ orientation:
a promoter that is functional in plants;
operatively linked to said promoter, a DNA sequence encoding a dominant-negative mutant of a plant DnaJ-like protein wherein the mutant interacts with a viral component, or a recombinant antibody that is specific for a plant DnaJ-like protein;
a termination signal; and
b) transferring the vector to the plant cell.

28. A transgenic plant cell or transgenic plant with increased virus resistance produced by the method of claim 2.

29. A transgenic plant cell or plant with increased virus resistance, wherein the expression of a DnaJ-like protein that interacts with a virus component is substantially prevented in the plant.

30. The transgenic plant cell or plant of claim 29, wherein the plant DnaJ-like protein is encoded by SEQ ID NO. 1, SEQ ID NO. 2 or SEQ ID NO. 3.

31. The transgenic plant cell or plant of claim 29, wherein the plant DnaJ-like protein is at least 30% homologous to SEQ ID NO. 4, SEQ ID NO. 5 or SEQ ID NO. 6.

32. A transgenic plant cell or plant with increased virus resistance, wherein a dominant-negative mutant of a plant DnaJ-like protein that interacts with a virus component, or an antibody specific for a DnaJ-like protein is expressed in the plant cell or the plant.

33. A transgenic plant cell or plant with increased virus resistance, wherein a dominant-negative mutant of a DnaJ-like protein that interacts with a virus component is expressed in the plant cell or the plant.

34. The transgenic plant cell or plant of claim 33, wherein the plant DnaJ-like protein is encoded by SEQ ID NO. 1, SEQ ID NO. 2 or SEQ ID NO. 3.

35. The transgenic plant cell or plant of claim 33, wherein the plant DnaJ-like protein is at least 40% homologous, at least 50% homologous, at least 60% homologous, at least 70% homologous, at least 80% homologous, or at least 90% homologous to SEQ ID NO. 4, SEQ ID NO. 5 or SEQ ID NO. 6.

36. The transgenic plant cell or plant of claim 33, wherein a protein is expressed as a dominant-negative mutant of a DnaJ-like protein in which the J-domain is deleted.

37. The transgenic plant cell or plant of claim 32, wherein an N-terminal deletion mutant of a plant DnaJ-like protein is expressed as a dominant-negative mutant in which the J-domain is deleted, and wherein said deleting mutant of a plant DnaJ-like protein is encoded by SEQ ID NO. 1, SEQ ID NO. 2 or SEQ ID NO. 3.

38. A transgenic plant cell or plant with increased virus resistance, wherein the J-domain is overexpressed.

39. A virus resistant plant cell or plant, comprising a mutation in a coding and/or regulatory sequence of a gene for a plant DnaJ-like protein, wherein said mutation causes a modulation of the expression of the protein or its binding behavior to a viral protein or an endogenous cellular binding partner.

40. The virus resistant plant cell or plant of claim 39, wherein a dominant-negative mutant of a plant DnaJ-like protein is expressed which is not able to interact with a viral component or a cellular interaction partner of a plant DnaJ-like protein.

41. The virus resistant plant cell or plant of claim 39, wherein said plant cell or plant is produced by the “TILLING” method.
